(19) United States
(12) Patent Application Publication
(10) Pub. No.: US 2024/0368551 A1
(43) Pub. Date: Nov. 7, 2024

(54) MEDIA FORMULATION FOR PRESERVING BIOLOGICAL SAMPLES AND METHOD FOR PRESERVING BIOLOGICAL SAMPLES

(71) Applicant: SINAI HEALTH SYSTEM, Toronto (CA)

(72) Inventors: Adele CHANGOOR, Scarborough (CA); Sarah Linda ALOI, Woodbridge (CA); Marc Daniel GRYNPAS, Toronto (CA)

(21) Appl. No.: 18/653,210

(22) Filed: May 2, 2024

Related U.S. Application Data
(60) Provisional application No. 63/463,451, filed on May 2, 2023.

(51) Int. Cl. C12N 5/077

(52) U.S. Cl. CPC C12N 5/0655; C12N 2500/32; C12N 2500/34; C12N 2500/60

(57) ABSTRACT

The specification provides an improved media formulation to enhance the viability of chondrocytes in stored biological samples. The formulation includes Dulbecco's Modified Eagle Medium (DMEM), doxycycline, and hyaluronic acid, in concentrations sufficient to maintain the chondrocytes' viability significantly longer than DMEM alone. This formulation allows biological samples containing chondrocytes to retain high viability rates for extended periods to be suitable for transplant purposes, such as in osteochondral tissue transplantation.

### CROSS-REFERENCE TO RELATED APPLICATION

This application claims the benefit of U.S. Provisional Patent Application No. 63/463,451 entitled “DEVELOPMENT OF A NOVEL STORAGE PROTOCOL FOR FRESH OSTEOCHONDRAL ALLOGRAFT PRESERVATION” filed May 2, 2023, the entire contents of which are incorporated herein by reference.

### FIELD

The present specification is directed to media formulations for preserving tissue, and in particular, osteochondral allografts.

### BACKGROUND

Osteochondral allograft transplantation is a surgical technique used to treat large cartilage focal lesions. The transplantation process involves resection and replacement of degraded cartilage with osteochondral grafts (consisting of bone and cartilage) harvested from a cadaveric donor. Success is dependent upon maintaining a minimum 70% chondrocyte viability within the donor tissue compared to fresh control. One storage method is Lactated Ringer's Solution supplemented with the antibiotics cefazolin and bacitracin, allowing for a maximum storage period of 14 days.

US Patent Publication No. 20200260720A1 provides a media formulation comprising DMEM and doxycycline for storing porcine cartilage plugs, however the media formulation is similarly unsuitable for long-term storage with cell metabolism decreasing by about half by the third week. European Patent Publication No. 3174565B1 describes several additives for media solutions, including N-acetyl-L-cysteine and hyaluronic acid, but cell viability declines below 70% by the third week.

### SUMMARY

An aspect of the specification provides a media formulation for preserving a biological sample comprising chondrocytes. The media formulation includes Dulbecco's Modified Eagle Medium (DMEM), doxycycline, and hyaluronic acid. The doxycycline and sodium hyaluronate are present in the media formulation in amounts effective to prolong the viability of chondrocytes in a biological sample maintained in the media formulation.

In some examples, the sodium hyaluronate has a molecular weight between about 1500 and about 2000 kDa.

In some examples, the sodium hyaluronate comprises about 0.1 percent of the media formulation by volume.

In some examples, the DMEM comprises between about 0 g/L and about 5 g/L glucose. In other examples, the DMEM comprises about 1 g/L glucose.

In some examples, the DMEM comprises L-glutamine.

In some examples, the DMEM comprises sodium pyruvate.

In some examples, the doxycycline comprises about 1 μg/mL of the media formulation. In other examples, the doxycycline comprises about 5 μg/mL of the media formulation.

In some examples, the doxycycline and sodium hyaluronate are present in the media formulation in amounts effective to maintain about 70% chondrocyte viability after a storage period of about 56 days.

A further aspect of the specification provides a method of preserving a biological sample. The method includes contacting a biological sample that includes chondrocytes with a media formulation that includes Dulbecco's Modified Eagle Medium (DMEM), doxycycline, and hyaluronic acid. The DMEM, doxycycline, and hyaluronic acid are present in amounts sufficient to prolong the viability of the chondrocytes. The method further includes maintaining the biological sample in the media formulation at a storage temperature between about 1° and about 12° C. At least 70% of chondrocytes in the biological sample remain viable after storage in the media formulation for a period of about 56 days.

In some examples, the storage temperature is about 10° C. In other examples, the storage temperature is about 4° C.

In some examples, the method further includes harvesting the biological sample from a cadaveric donor. The biological sample may be suitable for transplantation into a recipient. The biological sample may include an osteochondral tissue. In some examples, the method further includes assessing the viability of the chondrocytes, and if the viability exceeds a pre-determined threshold, transplanting the biological sample into the recipient. In some examples, transplanting the biological sample into the recipient comprises press-fitting the biological sample into a recipient site.

These together with other aspects and advantages which will be subsequently apparent, reside in the details of construction and operation as more fully hereinafter described and claimed, reference being had to the accompanying drawings forming a part hereof, wherein like numerals refer to like parts throughout.

### BRIEF DESCRIPTION OF THE DRAWINGS

Embodiments are described with reference to the following figures.

FIG. 1 is a schematic diagram of a method for preserving a biological sample, according to one embodiment.

FIG. 2 is a schematic diagram of a method for transplanting a biological sample, according to one embodiment.

FIG. 3 is a is a caudal view of the distal femur showing the source locations of the cartilage samples, according to Example 1.

FIG. 4 is a graph of the excitation and emission spectra for calcein and ethidium homodimer-1.

FIG. 5A is a confocal microscopy image, according to Example 1.

FIG. 5B is a confocal microscopy image, according to Example 1.

FIG. 5C is a confocal microscopy image, according to Example 1.

FIG. 5D is a confocal microscopy image, according to Example 1.

FIG. 6A is a confocal microscopy image, according to Example 1.

FIG. 6B is a confocal microscopy image, according to Example 1.

FIG. 6C is a confocal microscopy image, according to Example 1.

FIG. 6D is a confocal microscopy image, according to Example 1.

FIG. 7A is a graph of absolute cell viability versus storage time, according to Example 1.

FIG. 7B is a graph of normalized cell viability versus storage time, according to Example 1.

FIG. 8A is a microscopy image, according to Example 1.

FIG. 8B is a microscopy image, according to Example 1.

FIG. 8C is a microscopy image, according to Example 1.

FIG. 8D is a microscopy image, according to Example 1.

FIG. 9A is a microscopy image, according to Example 1.

FIG. 9B is a microscopy image, according to Example 1.

FIG. 9C is a microscopy image, according to Example 1.

FIG. 9D is a microscopy image, according to Example 1.

FIG. 10 is a schematic diagram of the experimental design for Examples 2 to 5.

FIG. 11 is a schematic diagram of the experimental design for Example 2.

FIG. 12A is a graph of absolute cell viability, according to Example 2.

FIG. 12 B is a graph of normalized cell viability, according to Example 2.

FIG. 13A is a microscopy image, according to Example 2.

FIG. 13B is a microscopy image, according to Example 2.

FIG. 13C is a microscopy image, according to Example 2.

FIG. 13D is a microscopy image, according to Example 2.

FIG. 14A is a microscopy image, according to Example 2.

FIG. 14B is a microscopy image, according to Example 2.

FIG. 14C is a microscopy image, according to Example 2.

FIG. 14D is a microscopy image, according to Example 2.

FIG. 15A is a microscopy image, according to Example 2.

FIG. 15B is a microscopy image, according to Example 2.

FIG. 16 is a graph of absolute cell viability versus storage time, according to Example 2.

FIG. 17A is a microscopy image, according to Example 2.

FIG. 17B is a microscopy image, according to Example 2.

FIG. 17C is a microscopy image, according to Example 2.

FIG. 17D is a microscopy image, according to Example 2.

FIG. 18A is a microscopy image, according to Example 2.

FIG. 18B is a microscopy image, according to Example 2.

FIG. 18C is a microscopy image, according to Example 2.

FIG. 18D is a microscopy image, according to Example 2.

FIG. 19A is a graph of RFU/mg versus storage time, according to Example 2.

FIG. 19B is a graph showing mean glycosaminoglycan, according to Example 2.

FIG. 20 is a schematic diagram of the experimental design for Example 3.

FIG. 21 is a graph of cell viability versus storage time, according to Example 3.

FIG. 22 is a graph of cell viability versus storage time, according to Example 3.

FIG. 23 is a schematic diagram of the experimental design of Example 4.

FIG. 24A is a table of metabolic activity, according to Example 4.

FIG. 24B is a graph of metabolic activity, according to Example 4.

FIG. 25A is a table of metabolic activity, according to Example 4.

FIG. 25B is a graph of metabolic activity, according to Example 4.

FIG. 26 is a microscopy image, according to Example 5.

FIG. 27 is a microscopy image, according to Example 5.

FIG. 28 is a microscopy image, according to Example 5.

FIG. 29 is a microscopy image, according to Example 3.

FIG. 30A is a table of DNA content, according to Example 5.

FIG. 30B is a graph of DNA content, according to Example 5.

### DETAILED DESCRIPTION

### Table of Abbreviations

The following abbreviations are used herein:

| | |
|---|---|
| CA | calcein AM |
| DMEM | Dulbecco's Modified Eagle Medium |
| DOX | doxycycline hyclate |
| DOX1/5/10 | doxycycline hyclate 1/5/10 μg/mL |
| ECM | extracellular matrix |
| EDTA | ethylenediaminetetraacetic acid |
| EtHD | ethidium homodimer-1 |
| GAG | glycosaminoglycan |
| HA | Hyaluronic acid |
| LFC | lateral femoral condyle |
| LRS | Lactated Ringer's solution |
| MFC | medial femoral condyle |
| OCAT | osteochondral allograft transplantation |
| PBS | phosphate buffered saline |
| PRG4 | proteoglycan-4 |
| RFU | relative fluorescence units |
| TUNEL | terminal deoxynucleotidyl transferase dUTP nick-end |
| | labeling |

### Definitions

“About” herein refers to a range of ±20% of the numerical value that follows. In one example, the term “about” refers to a range of ±10% of the numerical value that follows. In one example, the term “about” refers to a range of ±5% of the numerical value that follows.

“Hyaluronic acid” herein refers to the polysaccharide sometimes known as hyaluronan, having the molecular formula

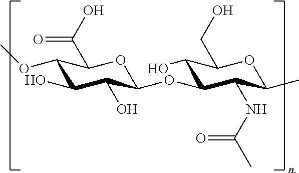

“Sodium hyaluronate” herein refers to a glycosaminoglycan which is found in various connective tissues of humans. Sodium hyaluronate is the sodium salt of hyaluronic acid.

“Doxycycline” herein refers to a tetracycline antibiotic having the molecular formula

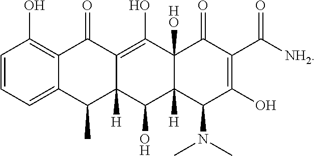 [SMILES: C[C@H]1c2cccc(O)c2C(=O)C2=C(O)[C@]3(O)C(=O)C(C(N)=O)=C(O)[C@@H](N(C)C)[C@@H]3[C@@H](O)[C@@H]21]

Doxycycline is sometimes referred to by the tradenames Doxy™, Doryx™, and Vibramycin™.

“Osteochondral tissue” herein refers to a tissue found at synovial joint surfaces which is composed of articular cartilage and bone.

### Media Formulation and Method of Preserving Biological Samples

The present specification provides an improved media formulation and method for preserving biological samples. The media formulation and method can maintain cell viability and metabolism at levels that exceed the performance of prior art media and double the duration that tissues meet or exceed the 70% viability threshold for transplantation. In contrast to US Patent Publication No. 20200260720A1, which provides a culture media comprising DMEM and doxycycline, the media formulation described herein includes hyaluronic acid, which greatly extends the chondrocyte viability to at least 56 days, and in some examples, 63 days. While hyaluronic acid has been previously used in storage media, it has not been combined with doxycycline and DMEM, and is generally overlooked in favor of other additives such as antioxidants (see for example, European Patent Publication No. 3174565B1 which shows that hyaluronic acid is relatively ineffective at maintaining cell viability). As will be described herein in greater detail, the synergistic combination of DMEM, doxycycline, and hyaluronic acid outperforms the results observed when DMEM is used only with doxycycline or only with hyaluronic acid.

The media formulation comprises Dulbecco's Modified Eagle Medium (DMEM). The DMEM may include one or more suitable components, including but not limited to, amino acids, sugars, vitamins, and salts. Suitable examples of amino acids include, but are not limited to glycine, L-arginine hydrochloride, L-cystine 2HCl, L-glutamine, L-histidine hydrochloride-H2O, L-Isoleucine, L-Leucine, L-Lysine hydrochloride, L-Methionine, L-phenylalanine, L-serine, L-threonine, L-tryptophan, L-tyrosine disodium salt dihydrate, and L-valine. Suitable examples of vitamins include, but are not limited to choline chloride, D-calcium pantothenate, folic acid, niacinamide, pyridoxine hydrochloride, riboflavin, thiamine hydrochloride, and i-inositol. Suitable examples of salts include, but are not limited to calcium chloride, ferric nitrate, magnesium sulfate, potassium chloride, sodium bicarbonate, sodium chloride, sodium pyruvate, and sodium phosphate monobasic.

A number of suitable DMEM solutions may be commercially available. In some examples, the DMEM is a low glucose DMEM comprising about 1 g/L glucose, L-glutamine, and sodium pyruvate.

The media formulation further comprises a tetracycline antibiotic, and in particular examples, the media formulation comprises doxycycline (DOX). Doxycycline inhibits matrix metalloproteinases which degrade the cartilage extracellular matrix, thereby preserving the extracellular matrix in the biological sample. Furthermore, doxycycline can enhance proliferation of chondrocytes and disrupt the terminal differentiation of chondrocytes.

The doxycycline may be present in the media formulation in an amount effective to reduce apoptosis of chondrocytes in the biological sample. In particular examples, the doxycycline is present in an amount effective to prolong the viability of chondrocytes as compared to the viability of chondrocytes stored in DMEM alone. In even more particular examples, the doxycycline is present in an amount effective to prolong the viability of chondrocytes as compared to the viability of chondrocytes stored in DMEM and doxycycline. In even more particular examples, the doxycycline is present in an amount effective to prolong the viability of chondrocytes as compared to the viability of chondrocytes stored in DMEM and hyaluronic acid.

The concentration of doxycycline may be between about 0 μg/mL and about 20 μg/mL, and in particular examples, the concentration is about 5 μg/mL or less. In some examples, the concentration of doxycycline is about 1 μg/mL. In some examples, the concentration of doxycycline is about 2 μg/mL. In some examples, the concentration of doxycycline is about 3 μg/mL. In some examples, the concentration of doxycycline is about 4 μg/mL. In some examples, the concentration of doxycycline is about 5 μg/mL.

The media formulation further comprises hyaluronic acid (HA). The hyaluronic acid may be provided in any suitable form including sodium hyaluronate, hydrolyzed hyaluronic acid, and sodium acetylated hyaluronate. These terms may be used interchangeably herein, and any description of hyaluronic acid may be similarly applied to sodium hyaluronate, hydrolyzed hyaluronic acid, and sodium acetylated hyaluronate.

The hyaluronic acid may have an average molecular weight between about 1 kDa and about 20,000 kDa. In some examples, the hyaluronic acid is a high molecular weight (HMW) hyaluronic acid having an average molecular weight greater than 800 kDa. In other examples, the hyaluronic acid has an average molecular weight between about 1,000 kDa and about 7,000 kDa. In further examples, the hyaluronic acid has an average molecular weight between about 1,500 kDa and about 2,000 kDa. The molecular weight of the hyaluronic acid may be selected to mimic the biological environment from which the biological sample was obtained, and in particular examples, the hyaluronic acid may be selected to mimic articular synovial fluid. Human synovial fluid contains hyaluronic acid with a very high molecular mass. Almost all of the hyaluronic acid in human synovial fluid is greater than about 1,000 kDa in size, and most of the hyaluronic acid is 2,500 to 7,000 kDa in size.

The hyaluronic acid may be present in the media formulation in an amount effective to reduce apoptosis of chondrocytes in the biological sample. In particular examples, the hyaluronic acid is present in an amount effective to prolong the viability of chondrocytes as compared to the viability of chondrocytes stored in DMEM alone. In even more particular examples, the hyaluronic acid is present in an amount effective to prolong the viability of chondrocytes as compared to the viability of chondrocytes stored in DMEM and doxycycline. In even more particular examples, the hyaluronic acid is present in an amount effective to prolong the viability of chondrocytes as compared to the viability of chondrocytes stored in DMEM and hyaluronic acid.

The hyaluronic acid may comprise between about 0 percent and about 2 percent of the media formulation by volume. In some examples, the hyaluronic acid comprises 0.01 percent of the media formulation by volume. In some examples, the hyaluronic acid comprises 0.05 percent of the media formulation by volume. In some examples, the hyaluronic acid comprises 0.1 percent of the media formulation by volume. In some examples, the hyaluronic acid comprises 0.2 percent of the media formulation by volume. In some examples, the hyaluronic acid comprises 0.5 percent of the media formulation by volume. In some examples, the hyaluronic acid comprises 1 percent of the media formulation by volume. In some examples, the hyaluronic acid comprises 2 percent of the media formulation by volume.

The hyaluronic acid may inhibit inflammation and/or apoptosis in the biological sample, and in particular, the chondrocytes. As will be explained in greater detail herein with respect to the Examples, hyaluronic acid may decrease apoptosis in the outer layers of the biological sample, and particularly the articular surface. These outer layers are subject to mechanical damage during an allograft procedure and are therefore particularly vulnerable to cell damage and cell death.

The media formulation may comprise one or more other additives. The additive may include, but is not limited to, an additional antibiotic, an antimycotic, a buffer, a growth factor, an antioxidant, a vitamin, a co-factor, a salt, a cryoprotectant, a cytokine, an enzyme, an amino acid, a sugar, a lipid, and a hormone. Specific non-limiting examples of additives include penicillin, streptomycin, HEPES ((4-(2-hydroxyethyl)-1-piperazineethanesulfonic acid)), dimethyl sulfoxide, glycerol, glucose, calcium, magnesium, perfluorocarbons, dexamethasone, ascorbate 2-phosphate, L-proline, sodium pyruvate, TGF-β, IGF-1, ZVAD-fmk, insulin-transferrin-selenium (ITS), insulin, insulin-like growth factors, transferrin, and selenous acid.

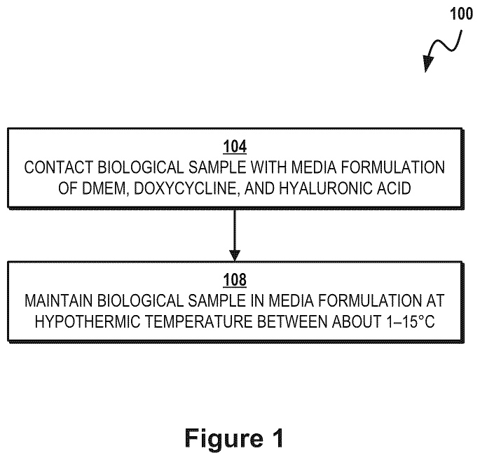

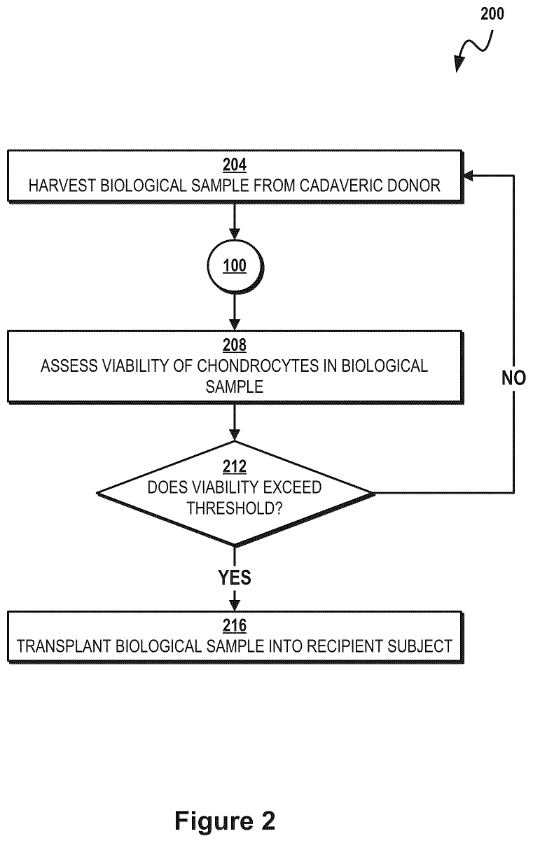

The media formulation may be used to preserve a biological sample, as described in greater detail with respect to FIGS. 1 and 2.

FIG. 1 shows a method for preserving a biological sample at 100.

Block 104 comprises contacting a biological sample with a media formulation comprising DMEM, doxycycline, and hyaluronic acid. For greater clarity, the media formulation may comprise any of the embodiments described above.

The biological sample may comprise an animal tissue, and more particularly, a mammalian tissue. The origin of the biological sample may be human, porcine, bovine, canine, caprine, equine, feline, galline, lapine, leporine, lupine, murine, ovine, porcine, vulpine, non-human primate origin, or the like. In preferred embodiments, the biological sample is human. The biological sample may be obtained from a living or cadaveric donor, a tissue culture, or a genetically-modified animal. Generally, the biological sample is an allograft which is selected according to the species of a recipient.

Generally, the biological sample includes chondrocytes. In particular, non-limiting examples, the biological sample includes an osteochondral tissue. The osteochondral tissue may be derived from a fibrous joint, a synovial joint, or a cartilaginous joint. Suitable joints include but are not limited to knee, shoulder, hip, elbow, ankle, spine, wrist, fingers, toes, and jaw. In particular examples, the osteochondral tissue is derived from the medial femoral condyle (MFC), lateral femoral condyle (LFC), medial trochlea, lateral trochlea, lateral intercondylar notch, patella, talus, iliac crest, tibial plateau, and the like.

The biological sample may comprise a cylindrical or dowel graft, a surface area graft, block graft, or shell graft. In specific non-limiting examples, the biological sample is dimensioned and shaped according to the recipient site.

Contacting the biological sample with the media formulation may comprise completely or partially submerging the biological sample in the media formulation. Contacting the biological sample with the media formulation may comprise spraying the biological sample with the media formulation. Contacting the biological sample with the media formulation may comprise coating the biological sample with the media formulation.

Block 108 comprises maintaining the biological sample in the media formulation at a storage temperature. The storage temperature may be a hypothermic storage temperature. In some examples, the storage temperature is between about 1° C. and about 15° C., and more particularly between about 2° C. to about 12° C. In some examples, the storage temperature is about 4° C. In other examples, the storage temperature is about 6° C. In further examples, the storage temperature is about 8° C. In yet further examples, the storage temperature is about 10° C. The storage temperature does not necessarily need to be constant, and in some examples, the storage temperature comprises a range of acceptable temperatures.

The biological sample may be maintained in the media formulation for any suitable storage period. In some examples, the storage period is 7 days. In some examples, the storage period is 14 days. In some examples, the storage period is 21 days. In some examples, the storage period is 28 days. In some examples, the storage period is 35 days. In some examples, the storage period is 42 days. In some examples, the storage period is 49 days. In some examples, the storage period is 56 days. In some examples, the storage period is 63 days. In some examples, the storage period is 70 days. In some examples, the storage period is 77 days. In some examples, the storage period is 84 days. In some examples, the storage period is 91 days. In some examples, the storage period is 98 days.

At least 50% of the chondrocytes in the biological sample may remain viable after storage in the media formulation for the storage period described above. In some examples, at least 70% of the chondrocytes in the biological sample remain viable after storage in the media formulation for the storage period described above.

As part of block 108, the media formulation may be changed once or periodically during the storage period. In examples where the media formulation is changed periodically, the media formulation may be changed daily, every other day, weekly, biweekly, monthly, or the like. The frequency may depend on the nature of the donor and the rate of cell metabolism. Biological samples derived from human tissue generally exhibit slower cell metabolism and therefore require less frequent changes. Changing the media formulation may inhibit bacteria growth and maintain the nutrient supply available to the biological sample.

The biological sample may be suitable for transplantation into a recipient.

FIG. 2 shows a method of transplanting a biological sample into a recipient at 200.

Block 204 comprises harvesting the biological sample from a cadaveric donor. The cadaveric donor may be human, porcine, bovine, canine, caprine, equine, feline, galline, lapine, leporine, lupine, murine, ovine, porcine, vulpine, non-human primate origin, or the like. The cadaveric donor may be selected according to the species of the recipient.

The biological sample is preserved in the media formulation according to the method 100 described above.

Block 208 comprises assessing the viability of the chondrocytes in the biological sample. The viability may be assessed with any suitable method known in the art, including but not limited to fluorescent dyes. In particular examples, cell viability is assessed by staining with Calcein-AM and Ethidium Homodimer-1.

Block 212 comprises determining whether the viability of the chondrocytes exceeds a pre-determined threshold. In some examples, the pre-determined threshold is 50%, however the threshold is not particularly limited. In other examples, the pre-determined threshold is 70%. In some examples, blocks 208 and 212 may be omitted, and the method 200 proceeds from block 204 to block 216.

In examples where the viability does not exceed the pre-determined threshold, the method 200 may return to block 204 and another biological sample is harvested.

In examples, where the viability meets or exceeds the pre-determined threshold, the method 200 may proceed to block 216.

Block 216 comprises transplanting the biological sample into the recipient. Any suitable grafting method may be used to perform block 216, including but not limited to the press-fit technique, the dowel technique, and the shell graft technique. In examples where the transplantation method is the press-fit technique, the block 216 includes press-fitting the biological sample into a recipient site.

Transplantation methods may inflict mechanical damage to the biological sample, which can detrimentally affect the success of the graft. In particular, the press-fit technique involves hammering the biological sample into the recipient site that is slightly smaller than the size of the biological sample. This can significantly damage the outer surfaces of the biological sample, causing damage to the ECM, and increasing the likelihood of apoptosis, necrosis, chondropoptosis, or autophagy. An advantage of including hyaluronic acid in the media formulation is that it can reduce the likelihood of cell death during storage, particularly on the surface of the biological sample, which can counteract the damaging effects of the transplantation process.

In view of the above, it will now be apparent that variants, combinations, and subsets of the foregoing embodiments are contemplated. For example, while the media formulation was discussed above in relation to osteochondral tissue, other tissues are contemplated.

It will now be apparent to a person of skill in the art that the present specification affords certain advantages over the prior art. While DMEM has been combined with doxycycline in the past, these storage media were not capable of maintaining chondrocyte viability beyond 21 days. With the further addition of hyaluronic acid, the media formulation described herein can maintain chondrocyte viability for 56 days, and in some examples more than 63 days. The hyaluronic acid mimics the native joint environment and provides optimal conditions to significantly extend the duration of cell viability at levels sufficient for transplantation. In particular, the hyaluronic acid reduces apoptosis at the surface of the tissue, which will increase chances of a successful allograft.

Additionally, the media formulation was found to have no detrimental effect on the extracellular matrix. The media formulation is highly effective for preservation of tissues and has the potential to significantly improve the supply of viable tissues for transplantation.

A further benefit of the media formulation is its simplicity and lower cost, which makes the formulation practical and economic for use in hospitals and tissue banks.

The media formulation and method will now be explained by way of example.

### Example 1

Example 1 examines the effects of the proposed formulation on chondrocyte viability and extracellular matrix (ECM) characteristics. The long-term storage of rabbit distal femurs was evaluated in a LRS standard media formulated according to the prior art solutions, and a DMEM-based media formulated according to the present specification.

### Methods

The left and right distal femurs from 10 male New Zealand White rabbits (3.71±0.4 kg) were assigned to storage in LRS standard media or the DMEM-based formulation for up to 56 days. The compositions of the two media formulations are shown in Table 1:

TABLE 1

| LRS Standard Media | DMEM-based Media |
|---|---|
| Lactated Ringer's Solution (1000 | DMEM (1.0 g/L glucose, with L- |
| mL bag) (Marchese Medical | glutamine & sodium pyruvate) |
| Supplies, Hamilton, Canada) | (Fisher Scientific ®, Markham, |
| | Canada) |
| Cefazolin (1 g/L) (Sigma- | Sodium hyaluronate (95% purity) |
| Aldrich ® Oakville, Canada) | (0.1% vol/vol), MW: 1500-2000 kDa |
| | (Fisher Scientific ®, Markham, |
| | Canada) |
| Bacitracin (50 000 U/L) (Sigma- | Doxycycline (5 μg/mL) (Sigma- |
| Aldrich ®, Oakville, Canada) | Aldrich ® Oakville, Canada) |

Samples from the left distal femur were stored in the LRS standard media, and samples from the right distal femur were stored in the DMEM-based media. The samples are summarized in Table 2:

TABLE 2

| | | |
|---|---|---|
| Storage Time | LRS Media | DMEM Media |

| | |
|---|---|
| 0 days | n = 4 |

| | | |
|---|---|---|
| 28 days | n = 3 | n = 3 |
| 42 days | n = 2 | n = 2 |
| 56 days | n = 2 | n = 2 |

| | |
|---|---|
| Total | n = 20 |

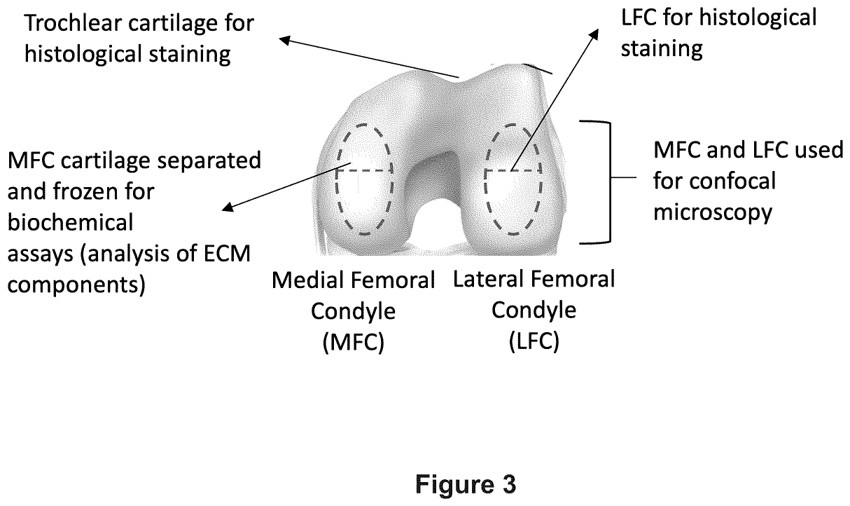

FIG. 3 is a caudal view of the distal femur showing the source locations of the cartilage samples.

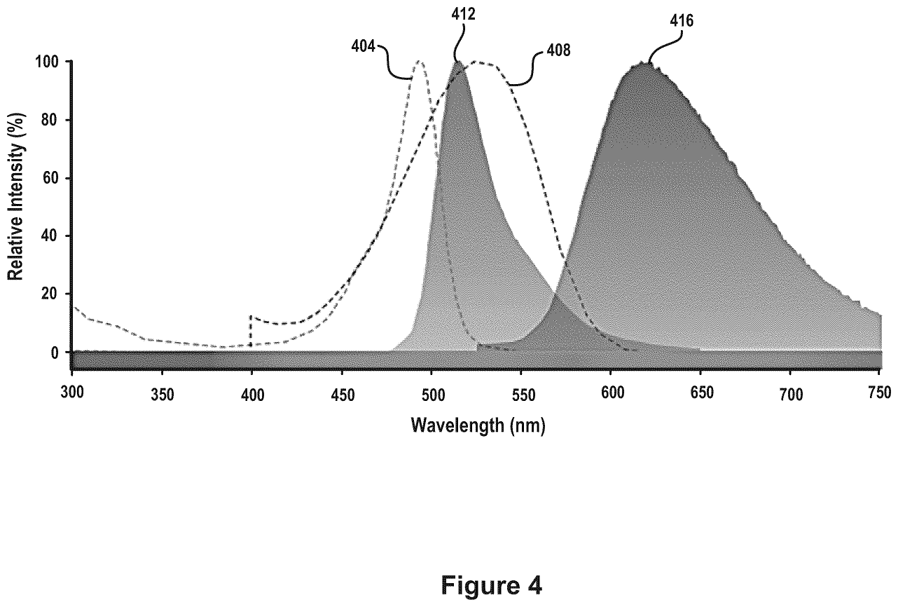

Chondrocyte viability was assessed by staining with Calcein-AM (Invitrogen®) (4 mM, 30 min incubation, emission: 515 nm), a cell-permeable dye that is converted to a green-fluorescent molecule by intracellular esterases, and Ethidium Homodimer-1 (2 mM, 50 min incubation, emission: 617 nm), a cell-impermeable dye that emits red fluorescence when bound to DNA. FIG. 4 shows the excitation spectra of Calcein 404, the excitation spectra of ethidium homodimer-1 408, the emission spectra of Calcein 412, and the emission spectra of ethidium homodimer-1 416.

Multiple cartilage pieces taken from the medial and lateral femoral condyles of each rabbit were stained and imaged on a Leica® SP8 confocal microscope at 10× objective at day 0 (N=7), day 28 (N=12), day 42 (N=8) and day 56 (N=8).

A custom-built program (MathWorks™ MATLAB R2022a) was used for analysis, identifying live cells (Calcein, stained green) and dead cells (ethidium homodimer-1, stained red) individually, as well as dual-stained cells (classified “dead” as penetration of ethidium homodimer-1 indicates decreased membrane integrity). Regions of interest selected for analysis were from sections of full thickness cartilage.

Osteochondral explants from the distal trochlea were fixed in 10% formalin, decalcified in 0.5 M EDTA (ethylenediaminetetraacetic acid) and embedded in paraffin wax. Sections (5 μm thick) were stained with Safranin-O/Fast Green or Toluidine Blue to qualitatively assess the ECM proteoglycan content.

Statistical analyses to compare chondrocyte viability at each storage time were done using Welch's unequal variance t-test to account for small sample sizes (Graphpad™ Prism 10.0.2).

### Results

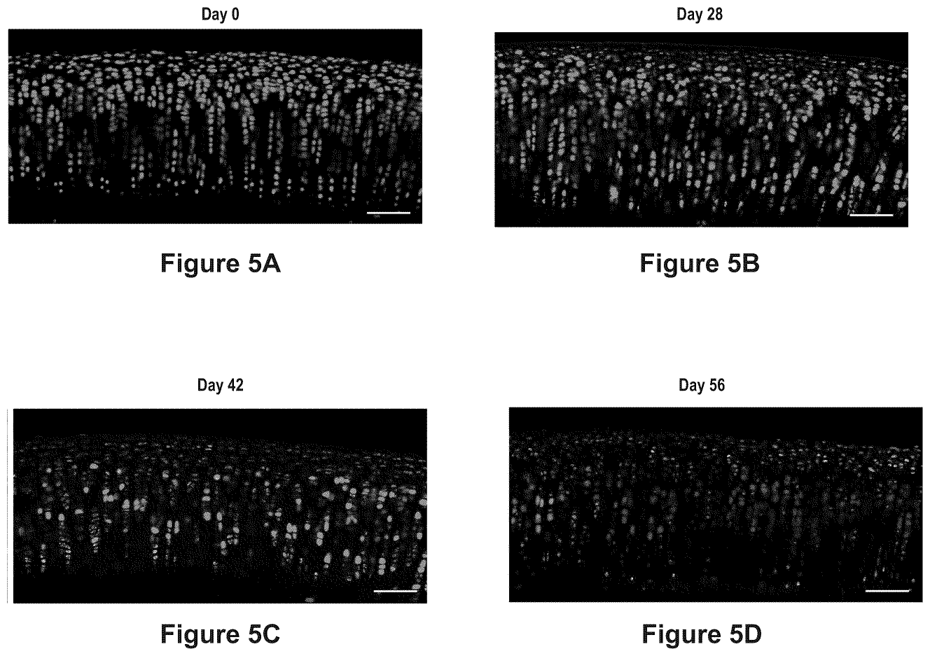

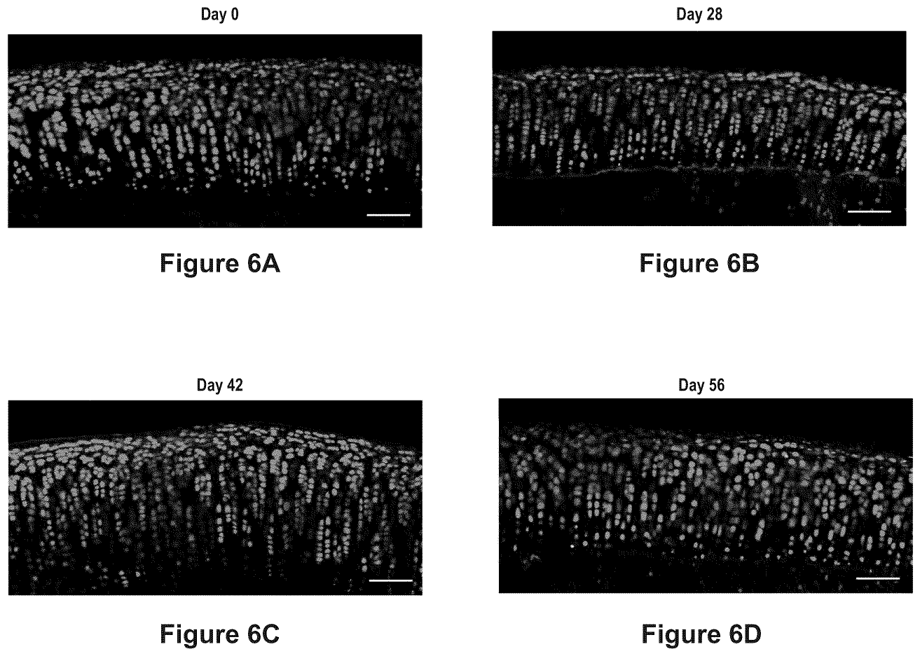

Representative confocal microscopy images displaying distribution of live (green, calcein-AM) and dead (red, ethidium homodimer-1) chondrocytes in sections taken from the medial femoral condyles are provided in FIGS. 5A-5D and 6A-6D. FIGS. 5A to 5D shows microscopy images of cells after storage in LRS standard media. FIGS. 6A to 6D show microscopy images of cells after storage in the DMEM-based media formulation. The images are oriented with the articular surface at the top and underlying subchondral bone at the bottom. The microscopy images of FIGS. 5A-5D and 6A-6D are taken of cross-sections from the medial femoral condyles. The scale bar represents 100 μm.

FIGS. 5A-5D and 6A-6D show that cell viability in rabbit cartilage was maintained throughout 56 days of storage in the DMEM-based media formulation. Further, the microscopy shows that cell death occurs first at the surface of the biological sample, and particularly the articular surface, and slowly spreads through the sample. FIGS. 6A-6D show that cell death is reduced in the DMEM-based media formulation, and this impact is more pronounced at the articular surface (top).

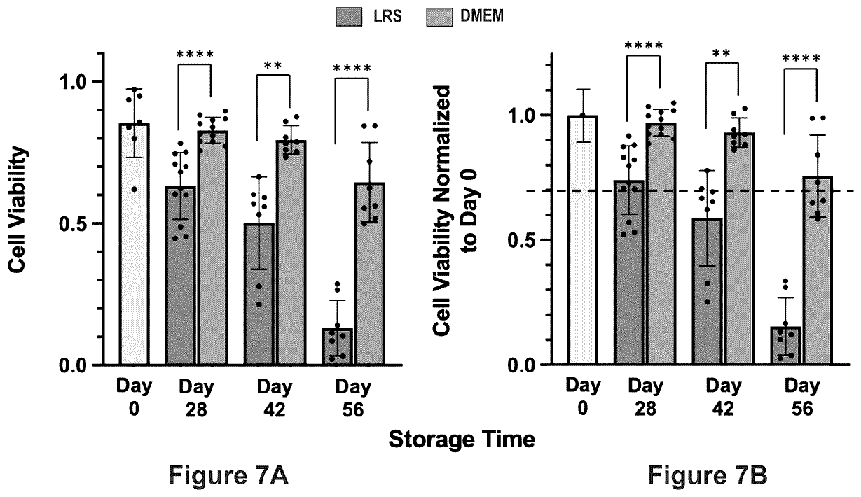

FIG. 7A and FIG. 7B are graphs showing the cell viability according to Example 1. FIGS. 7A and 7B show the cell viability in both medial and lateral femoral condyle samples after storage in LRS standard media and DMEM-based media for 0, 28, 42 and 56 days. FIG. 7A is a graph of the absolute cell viability shown as a fraction of total cell number. FIG. 7B is a graph of the cell viability normalized to Day 0. In FIGS. 7A an 7B, the dotted line represents 70% cell viability compared to Day 0, ** indicates p<0.01, and **** indicates p<0.0001.

Normalized to fresh control, cell viability in the DMEM-based media was measured to be 93.1% and 75.6% at day 42 and 56 compared to 58.7% and 15.4% in LRS storage (p<0.01, p<0.0001) (FIGS. 7A and 7B).

Absolute cell viability was maintained above 70% in the DMEM-based storage media for the entire 56-day storage period and fell below 60% by 42 days in LRS standard media (FIGS. 7A and 7B).

Compared to LRS standard media, absolute cell viability results display 29.3% more viable cells after 42 days and 51.5% after 56 days of storage in the DMEM-based media formulation.

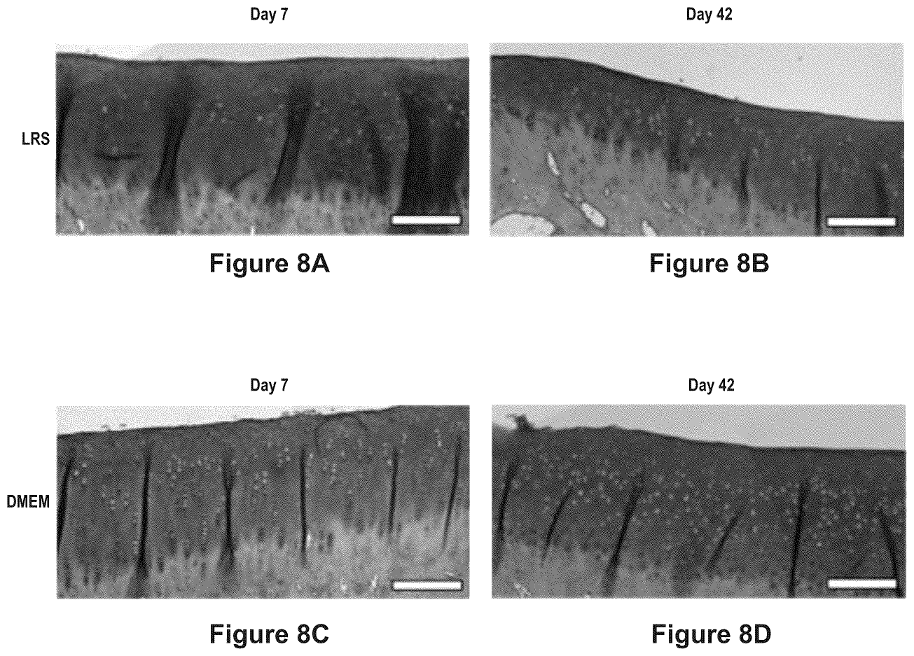

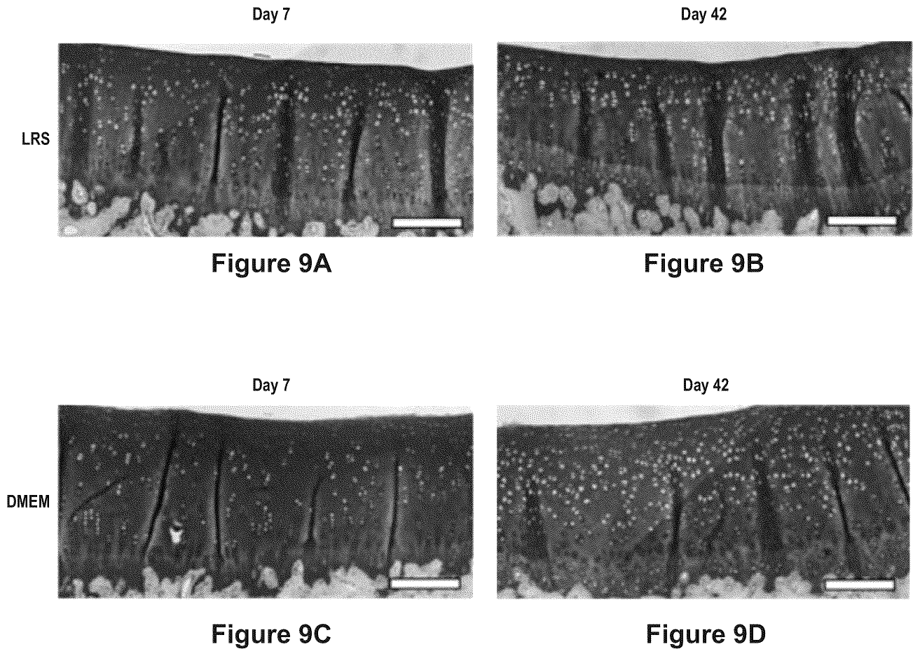

FIGS. 8A to 8D shows Safranin-O/Fast Green-Stained trochlea sections. FIGS. 9A to 9D show Toluidine Blue-Stained Trochlea Sections. Images acquired at 4× objective using an inverted brightfield microscope after 7-day and 42-day storage. The scale bar represents 500 μm. No significant qualitative differences were observed in Safranin-O or Toluidine Blue stain retention as storage time progressed, indicating no changes in the proteoglycan content of the ECM.

### DISCUSSION

The number of dead cells increased along the articular surface and towards the subchondral bone with prolonged storage under both conditions, however, this effect is more pronounced in cartilage stored in the LRS standard media (FIGS. 5A-5D and 6A-6D). The results suggest the ability of high molecular weight hyaluronic acid to provide protection and lubrication within the natural joint environment and maintain glycosaminoglycan (GAG) content in the extracellular matrix (ECM). Doxycycline is an antibiotic which has been shown to disrupt terminal differentiation of chondrocytes and inhibit degradation of ECM components. The anti-apoptotic effects of doxycycline may be contributing to the increased viability observed when compared to LRS standard media.

High variability was observed among day 0 fresh controls due to an increased dead cell count in one sample analyzed 24 hours after sacrifice, which contributed to the relatively high normalized data at earlier time points.

Histological staining indicated that both storage methods maintained proteoglycan content up to 42 days and may be due to rabbits retaining higher intrinsic repair abilities at maturity, indicating an opportunity for further testing on human or other animal tissues.

The DMEM-based media formulation maintained superior chondrocyte viability in rabbit tissues, specifically, above 90% and 70% of fresh controls for up to 42 and 56 days, respectively.

The results of Example 1 support further exploration of the DMEM-based storage protocol and contribute to efforts to increase the quality and quantity of human osteochondral tissues available for clinical use.

### Examples 2-5

Examples 2 to 5 were conducted using osteochondral tissues harvested aseptically from mature New Zealand rabbits (n=22, 3.67±0.14 kg) with the following specific objectives: (1) Determine the length of time during which osteochondral tissues can be stored in the DMEM-based media and maintain a minimum of 70% chondrocyte viability; (2) investigate the effects of doxycycline concentration on chondrocyte viability during prolonged storage; (3) evaluate the effect of using DMEM on chondrocyte metabolism during prolonged storage; and (4) investigate the effect hyaluronic acid on chondrocyte metabolism and cell apoptosis.

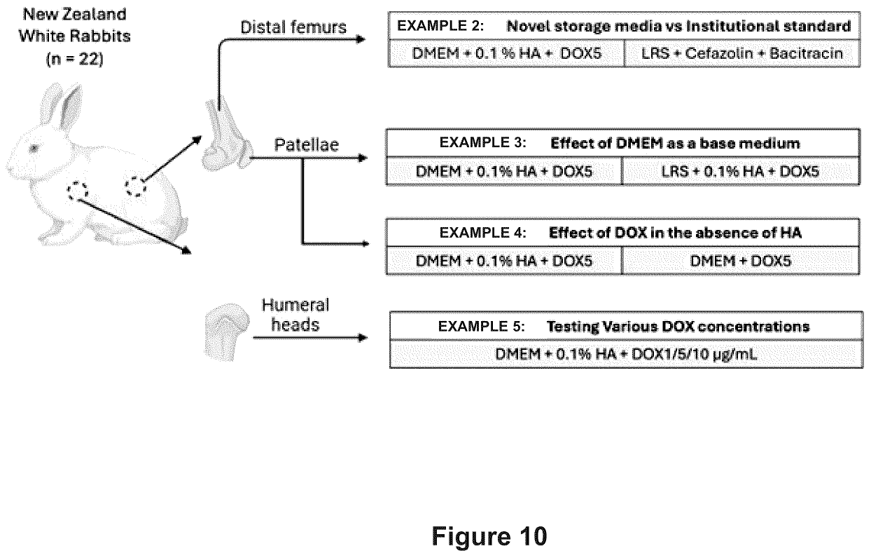

FIG. 10 is a schematic diagram showing the experimental design for Examples 2 to 5. Examples 2 to 5 use the distal femurs, patellae, and humeral heads from 22 mature New Zealand white rabbits. DOX1, DOX5, and DOX10 indicate doxycycline concentrations of 1, 5, and 10 μg/mL, respectively.

Table 3 shows group sizes for each experiment where n is the number of joints. Example 2 used distal femurs, Example 3 used humeral heads and both Example 4 and Example 5 used patellae.

TABLE 3

| Storage | EXAM- | EXAMPLE 3 | | | EXAM- | EXAM- |
|---|---|---|---|---|---|---|
| Time | PLE 2 | DOX 1 | DOX 5 | DOX 10 | PLE 4 | PLE 5 |
| Day 0 | n = 4 | n = 2 | | | n = 3 | n = 3 |
| Day 7 | n = 2 | — | — | — | — | — |
| Day 14 | — | — | — | — | n = 1 | — |
| Day 21 | n = 2 | — | — | — | — | — |
| Day 28 | n = 4 | — | — | — | n = 3 | n = 5 |
| Day 42 | n = 4 | n = 4 | n = 3 | n = 4 | — | — |
| Day 56 | n = 4 | n = 3 | n = 3 | n = 3 | — | — |
| Day 63 | n = 4 | — | — | — | — | — |

Statistical comparisons were made using Welch's unequal variance t-test, which accounts for small and unequal sample sizes and unequal group variance. Comparisons were made between storage conditions at each storage time and to identify differences between medial and lateral femoral condyles. Similarly, the Welch's unequal variance t-test was also used to determine the effect of doxycycline concentration on cell viability and the effects of DMEM and hyaluronic acid on metabolic activity.

### Example 2: Superior Chondrocyte Viability and Cartilage Quality Following Storage in the DMEM Media Compared to the LRS Standard

### Methods

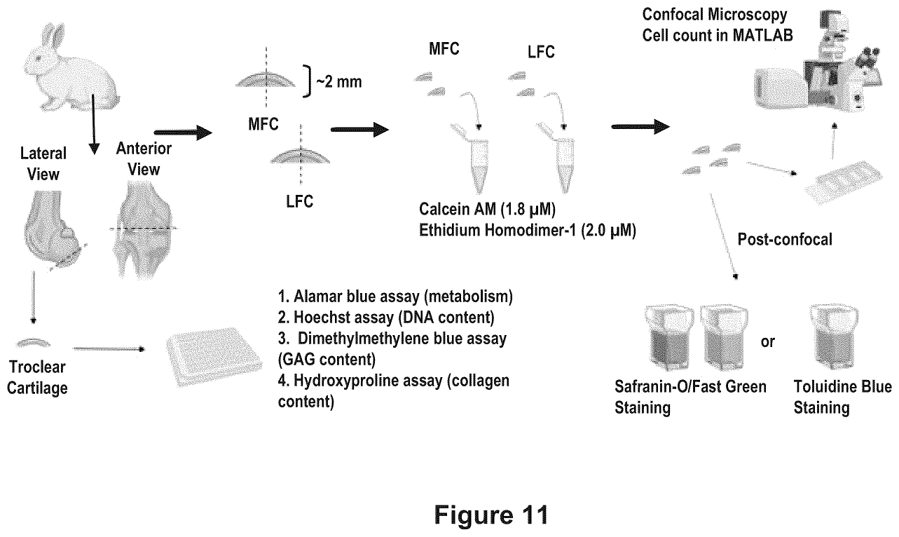

FIG. 11 is a schematic diagram of the methods for Example 2, including staining and imaging methods for the distal femur and subsequent analyses. Osteochondral samples were isolated from the medial (MFC) and lateral (LFC) femoral condyles, stained in Calcein AM and Ethidium Homodimer-1 and imaged on an inverted confocal microscope for live/dead quantification. Samples from the MFC and LFC were then fixed in formalin for histological staining in safranin-O/fast green or toluidine blue for proteoglycan content. Trochlear cartilage was separated from the bone and used for assessment of metabolic function using the Alamar blue assay. Trochlear cartilage was subsequently frozen and used for biochemical analyses to quantify extracellular matrix composition.

The distal femurs of 22 rabbits were isolated aseptically. The left distal femur of each rabbit was stored in 150 mL of the LRS standard media, consisting of Lactated Ringer's Solution (LRS), cefazolin (1 g/L) and bacitracin (5000 U/L), and the right distal femur was stored in the DMEM-based media, consisting of DMEM (1.0 g/L glucose, L-glutamine, sodium pyruvate), high molecular weight hyaluronic acid (0.1% vol/vol) and doxycycline (5 μg/mL). All samples were stored at 4° C., protected from light and subjected to weekly media changes. Tissue was stored for 0 (fresh controls, n=4), 7 (n=2), 21 (n=2), 28 (n=4), 42 (n=4), 56 (n=4) or 63 days (n=3) (±1 day), where n indicates the number of distal femurs per storage condition per time point.

Chondrocyte viability was assessed at the end of the assigned storage times using fluorescent dyes to quantify the number of live and dead cells in cartilage cross-sections. First, the medial (MFC) and lateral (LFC) femoral condyles (FIG. 11) were isolated using a low-speed precision saw (IsoMet, Buehler Ltd., Illinois, USA). Cross-sections, which included the full thickness of cartilage and 1-2 mm of underlying subchondral bone, were prepared by bisecting each condyle using a beveled flat blade (Blade #17, X-Acto™, Westerville, USA). Each tissue piece was then stained concurrently with 1.8 μM Calcein AM (CA) and 2 1.8 μM Ethidium homodimer-1 (EtHD) for 50 minutes. CA is a fluorescent dye (excitation: 495 nm, emission: 515 nm) that passes through all cell membranes and fluoresces green when it is cleaved by cellular esterases in the cytoplasm of live cells. Ethidium homodimer-1 (EtHD) is a fluorescent dye (excitation: 528 nm, emission: 617 nm) that binds to DNA and is membrane impermeable, meaning that it is only able to bind to cells with damaged cellular and nuclear membranes, resulting in dead or dying cells fluorescing red. Stained tissues were imaged using a 10× objective lens on a Leica SP8 inverted confocal microscope running LAS X™ software. Raw images were imported into a validated, custom MATLAB cell counting software (MathWorks™ MATLAB R2022a) where the number of live and dead cells, represented by green and red stained cells, respectively were quantified. Dual-stained cells were counted as dead, as penetration of EtHD indicates loss of nuclear membrane integrity and indicates a dying cell. Images from four cross-sections were obtained for each distal femur and included in data analysis. Imaging was completed within 2-3 hours of staining.

Following chondrocyte viability assessments, MFC and LFC tissue pieces underwent histological processing. Histological processing involved fixing tissue in 10% formalin for one week, followed by decalcification using 0.5 M ethylenediaminetetraacetic acid, and finally, infiltration and embedding in paraffin wax. Sections, 5 μm thick, were then stained with Safranin O/Fast green or Toluidine blue to visualize proteoglycan content and distribution on an inverted brightfield microscope.

Chondrocyte metabolism was assessed on cartilage obtained from the trochlea of each distal femur using the Alamar Blue assay, which quantifies metabolic activity through a water-soluble fluorometric indicator. The full thickness of cartilage, from the superficial to the deep zone, was isolated using a beveled flat blade (Blade #17, X-Acto™, Westerville, USA), hydrated in phosphate buffered saline solution for 15 minutes, and the wet weight measured. Cartilage pieces were then incubated in 10% Alamar Blue, a resazurin-based solution, for 3 hours at 37° C. Metabolically active cells reduce resazurin, which is blue in color, to resorufin, which is red in color and highly fluorescent. Fluorescence was measured in quadruplicate on a plate reader running EnSpire™ software (Perkin Elmer) using an excitation wavelength of 530-560 nm and emission at 590 nm. Results were reported as relative fluorescence units (RFU) normalized to the wet weight of the sample.

These trochlear tissue pieces were then frozen at −20° C. for biochemical assays to quantify proteoglycan content and chondrocyte number. Proteoglycan content, one of the two major constituents of the cartilage extracellular matrix, was assessed using the Dimethylmethylene Blue (DMMB) assay. This colormetric assay is used to quantify the amount of sulphated glycosaminoglycan (sGAG) contained in papain digested cartilage samples by mixing with 1,9-dimethylmethylene blue dye. DMMB dye binds to sGAG and undergoes a color change (metachromasia) from blue to purple upon binding. The sGAG-DMMB complex rapidly forms a precipitate. Bovine serum albumin in the GAG standard solution is used to stabilize the soluble sGAG-DMMB complex thereby allowing a number of samples to be evaluated simultaneously in a 96-well plate.

### Results

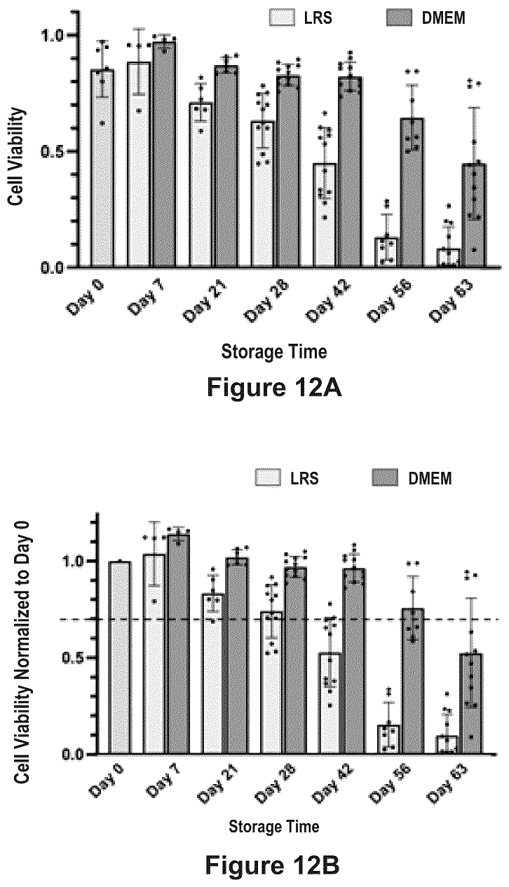

Chondrocyte viability evaluated in lapine osteochondral tissues was consistently higher after storage in the DMEM-based media formulation compared to the LRS standard media at all time points, with statistically significant differences detected at storages times of 21, 28, 42, 56 and 63 days (Table 4, FIG. 12). Table 4 shows the absolute and normalized average cell viability for the distal femurs over time. Absolute values were normalized to viability measured in fresh controls.

TABLE 4

| | | | | | | | |
|---|---|---|---|---|---|---|---|
| Storage | Day 0 | Day 7 | Day 21 | Day 28 | Day 42 | Day 56 | Day 63 |

Absolute Cell Viability Data

| | | | | | | | |
|---|---|---|---|---|---|---|---|
| LRS | 85.4% | 88.6% | 71.1% | 63.2% | 45.0% | 13.1% | 8.3% |
| DMEM | | 97.3% | 87.0% | 82.8% | 82.2% | 64.6% | 44.7% |

Normalized Cell Viability Data

| | | | | | | | |
|---|---|---|---|---|---|---|---|
| LRS | 100% | 104% | 83.3% | 74.1% | 58.7% | 15.4% | 9.7% |
| DMEM | | 114% | 102% | 97.0% | 93.1% | 75.6% | 52.4% |

FIG. 12A is a graph of absolute cell viability for the distal femurs over time. FIG. 12B is a graph of cell viability normalized to Day 0. Bars represent the mean and standard deviation. The dotted line represents the minimum 70% threshold for chondrocyte viability broadly accepted for fresh osteochondral allograft transplantation (OCAT). Statistical comparisons were made per storage time and the DMEM-based media was significantly higher (p<0.05) than the LRS standard media from day 21 to day 63 inclusive.

Absolute cell viability, averaged from both the LFC and MFC cartilage, indicated that at 28, 42, 56, and 63 days, cell viability was 83.8±4.5%, 77.4±11.4%, 73.6±16.6% and 44.7±24.2% after storage in the DMEM media formulation, respectively (Table 4, FIGS. 12A and 12B). In comparison, cell viability in samples preserved in the LRS standard media was 67.2±12.5%, 36.6±20.1%, 27.0±24.1% and 8.3±9.3% at days 28, 42, 56 and 63, respectively (Table 4, FIGS. 12A and 12B).

When normalized to fresh controls (85.4% viability at day 0), cell viability in the DMEM-based media was maintained above the 70% threshold for transplantation for up to 56 days of storage (FIGS. 12A and 12B) and was above 50% after 63 days of storage. More specifically, tissues stored in the DMEM-based media maintained viability at 98.2±5.3% at day 28, 90.7±13.3% at day 42 and 86.2%±19.4% at day 56, compared to fresh controls. In comparison, the LRS standard media maintained viability above the minimum 70% for only up to 28 days (74.1±1478.8±15%), and by days 42 and 56, the normalized cell viability was 52.7±1842.9±23.6% and 15.4±1231.7±28.2%, respectively (Table 4, FIGS. 12A and 12B).

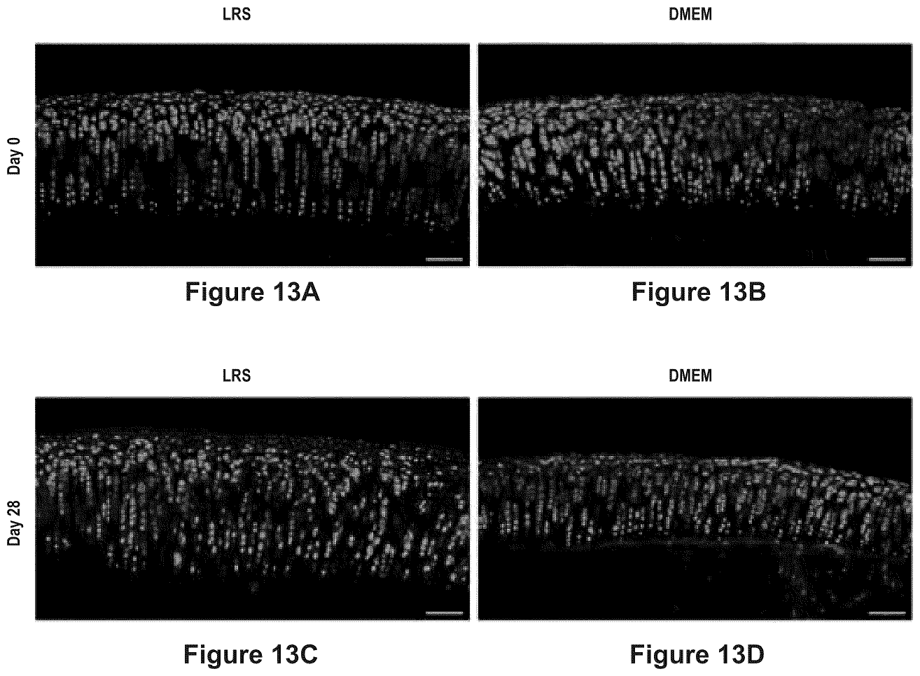

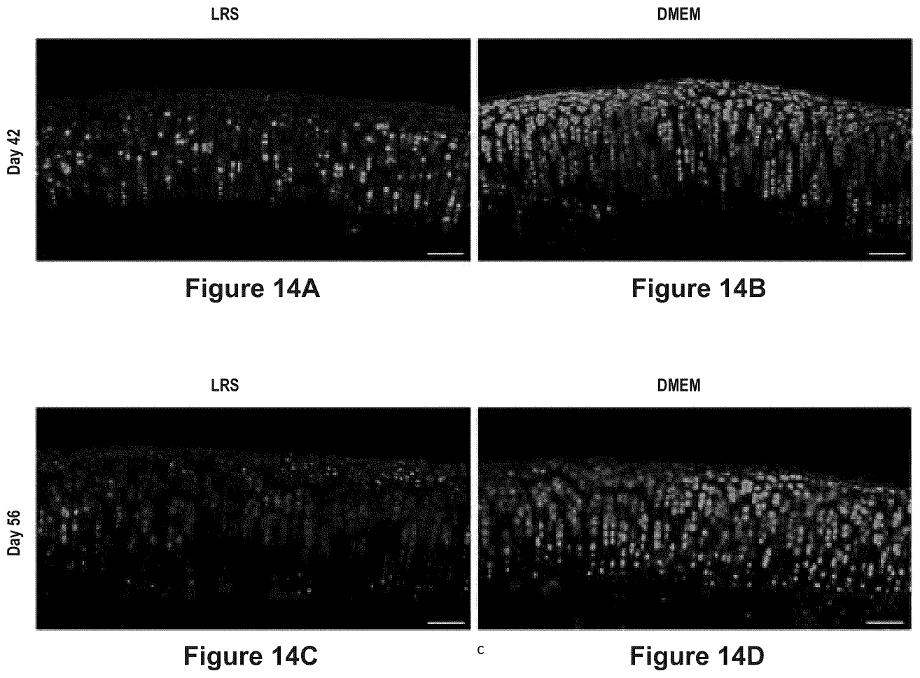

FIGS. 13A to 13D, 14A to 14D, 15A and 15B are representative confocal microscopy images of medial femoral condyles. The distribution of live and dead chondrocytes are shown by Calcein AM positive (green) and Ethidium Homodimer-1 EtHD positive (red) cells. Images are oriented with the articular surface at the top and underlying subchondral bone at the bottom. Scale bar represents 100 μm.

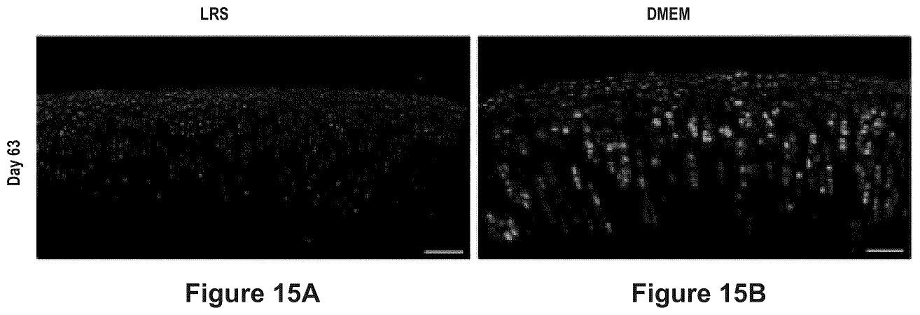

The confocal microscopy images were qualitatively analyzed to observe patterns in cell death occurring over time, as shown in FIGS. 13A to 13D, 14A to 14D, 15A and 15B. Samples stored in the LRS standard media, exhibited a pattern of chondrocyte death that began at the articular surface at 28 days and increased progressively toward the subchondral bone as storage time increased. In contrast, this pattern does not appear in samples stored in the DMEM-based media until Day 63 (FIG. 15B).

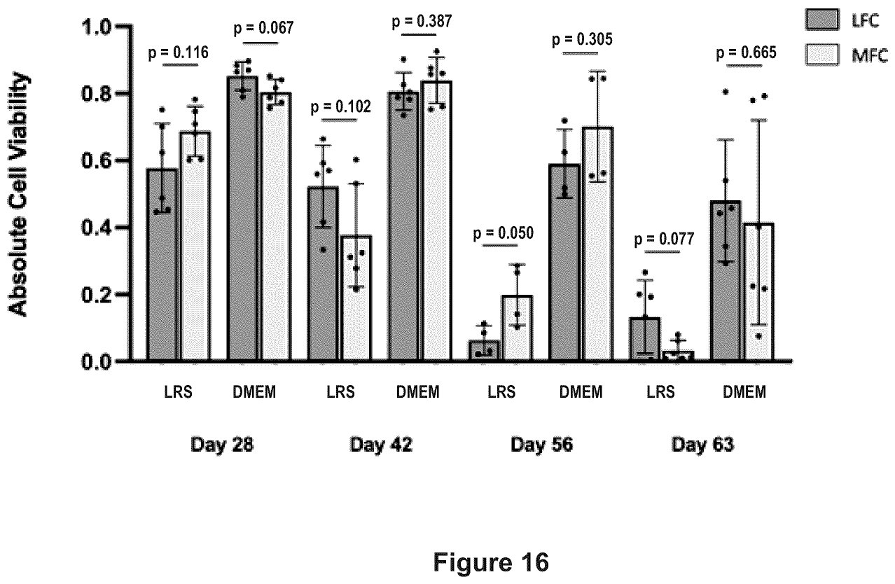

The chondrocyte viability data described above includes measurements from both the MFC and LFC (Table 4, FIGS. 12A and 12B). Prior to combining the data from these two joint surfaces, cell viability was examined for the MFC and LFC separately (FIG. 16). This analysis found that the average absolute cell viability was not statistically significantly difference (p≥0.05) between the joint surfaces at any time point. This finding was consistent between the DMEM-based media and the LRS standard media (FIG. 16). FIG. 16 is a graph showing the location-dependent chondrocyte viability of the distal femurs. Differences in cell viability between the lateral and medial femoral condyles were not statistically significant in both the LRS standard media and DMEM-based media from 28 to 63 days of storage (p≥0.05). The number of distal femurs included in this analysis were Day 28 (n=3), Day 42 (n=3), Day 63 (n=3), and Day 56 (n=2).

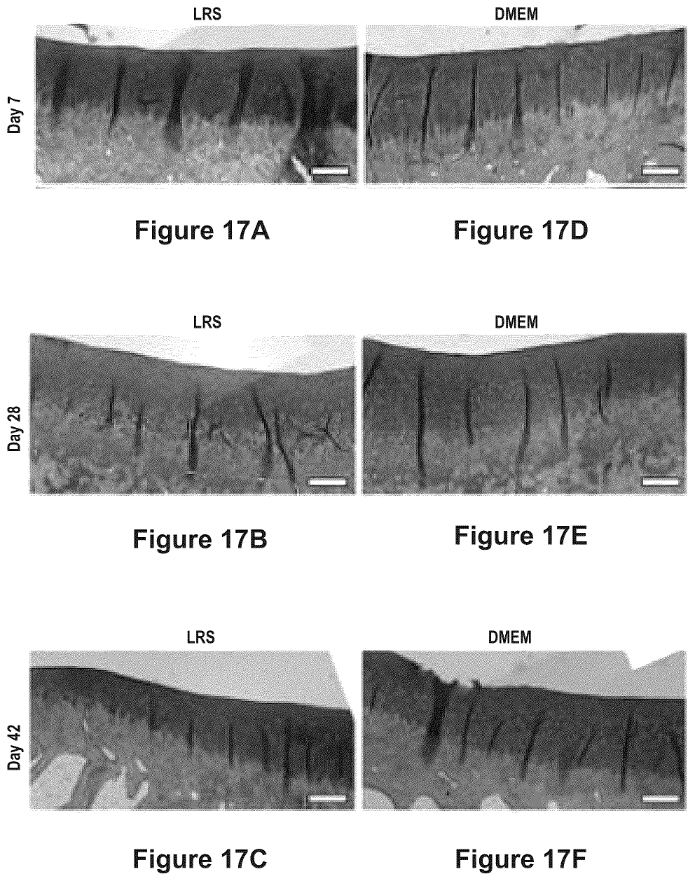

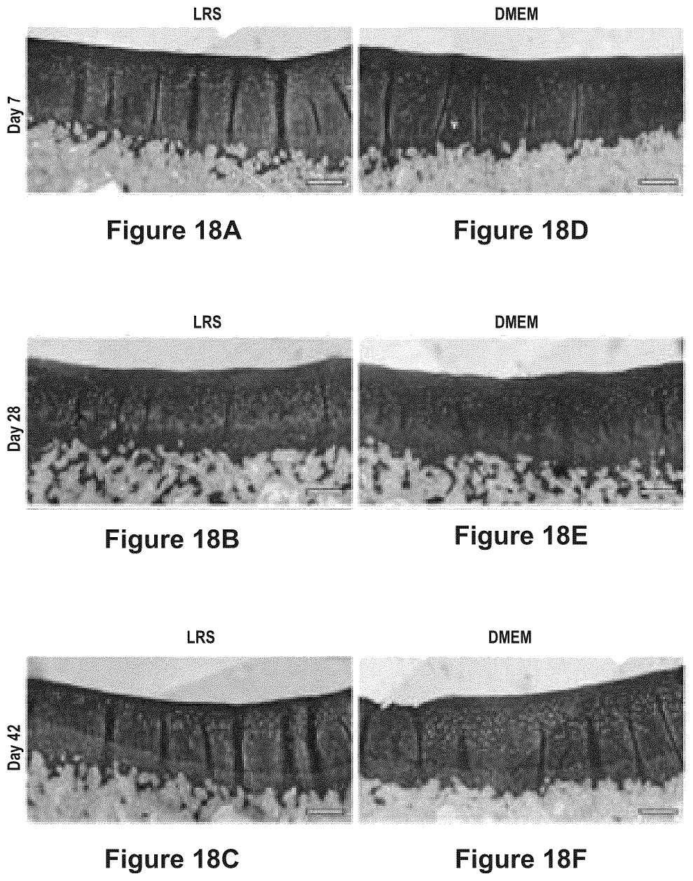

Histological sections revealed no meaningful differences in proteoglycan content, evaluated with either Safranin O/Fast green or toluidine blue, at any time point. This finding was consistent for both the DMEM media and LRS standard media. FIGS. 17A to 17F and FIGS. 18A to 18F show histological staining of cartilage obtained from the trochlea. Sections were stained with either Safranin O/fast green (FIGS. 17A to 17F) or Toluidine blue (FIGS. 18A to 18F) for the presence and distribution of proteoglycan within the cartilage extracellular matrix. Scale bars represents 500 μm.

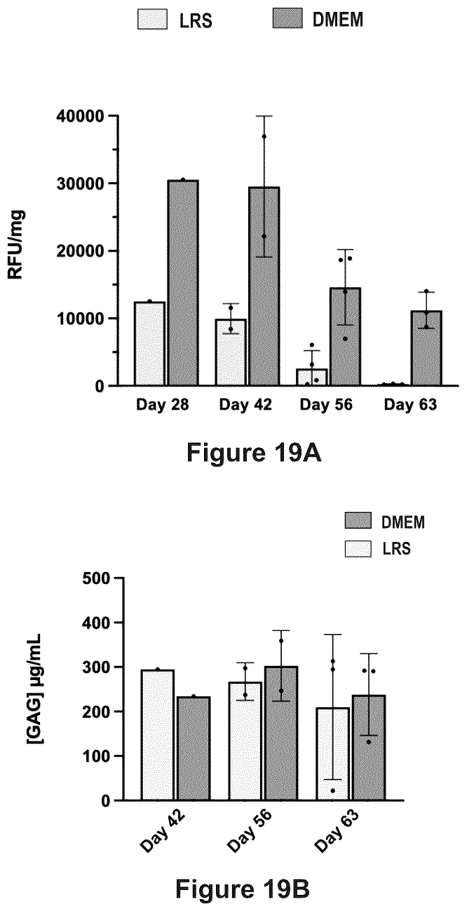

Metabolic activity was quantified using the Alamar Blue assay and samples stored in the DMEM-based media formulation 14,610.3 RFU/mg and 11,199.8 RFU/mg at day 56 and 63, respectively (FIG. 19A). These results were approximately 6 to 40 times higher than the metabolic activity detected in samples stored in the institutional standard, which averaged 2580.4 RFU/mg and 252.7 RFU/mg after 56 and 63 days of storage, respectively (Table 5, FIG. 19A). These results were at least 20 times higher than the metabolic activity detected in samples stored in the LRS standard media, which averaged 533.4 RFU/mg and 252.7 RFU/mg after 56 and 63 days of storage, respectively (Table 5).

TABLE 5
Metabolic Activity (RFU/mg)

| Storage Time | LRS | DMEM |
|---|---|---|
| Day 28 | 12534.6 | 30546.8 |
| Day 42 | 9969.9 | 29540.2 |
| Day 56 | 2580.4 | 14610.3 |
| Day 63 | 252.7 | 11199.8 |

Table 5 and FIG. 19A show chondrocyte metabolism during prolonged storage of distal femurs in the DMEM-based media or LRS standard media. Data are relative fluorescence units normalized to wet weight (RFU/mg) and bars are mean and standard deviation. Day 28 (n=1), day 42 (n=2), day 56 (n=4) and day 63 (n=3).

Proteoglycan content was assessed biochemically using the DMMB assay and no significant differences were detected between the DMEM media and LRS standard media (Table 6, FIG. 19B). FIG. 19B is a graph showing mean glycosaminoglycan (GAG) concentration after storage for 42, 56 or 63 days. Mean and standard deviation are indicated on the graph. No significant differences in GAG content were detected between the DMEM media and LRS standard media.

TABLE 6
Mean GAG Concentration (μg/mL)

| Storage Time | LRS | DMEM |
|---|---|---|
| Day 42 | 294.6 | 234.4 |
| Day 56 | 267.3 | 302.7 |
| Day 63 | 209.9 | 238.0 |

In summary, these experiments determined that the DMEM media could maintain a minimum of 70% chondrocyte viability compared to controls in osteochondral tissues stored up to 56 days, and that the DMEM media formulation exceeds 50% viability compared to controls after 63 days of storage. These experiments also showed that samples stored in the DMEM-based media exhibited metabolic activity approximately 20-fold that measured in samples stored in the LRS standard media, while both storage conditions maintained proteoglycan content in the cartilage extracellular matrix over time.

### Example 3: Lower Concentrations of Doxycycline Support Higher Chondrocyte Viability

### Methods

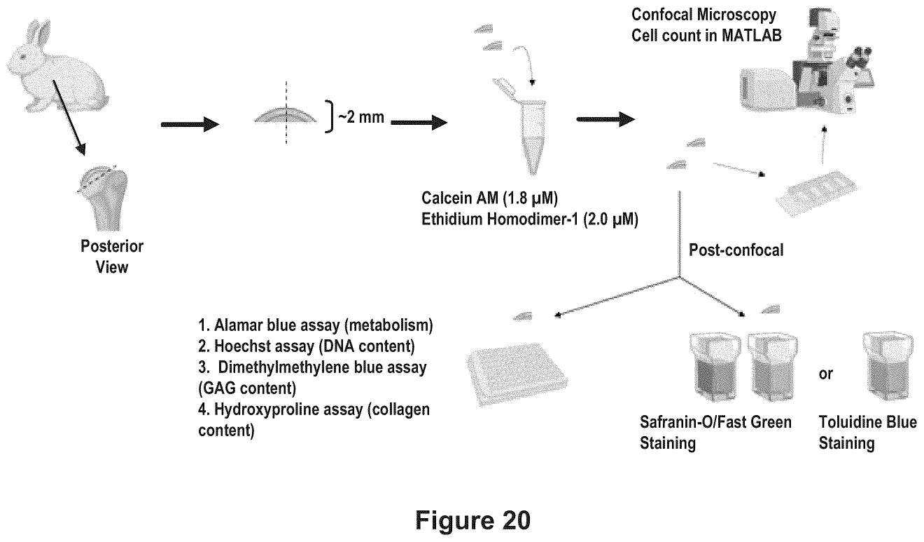

The humeral heads of 11 rabbits were isolated aseptically and stored in 40 mL of DMEM (1.0 g/L glucose, L-glutamine, sodium pyruvate), high molecular weight hyaluronic acid (0.1% vol/vol) and doxycycline at concentrations of either 1 μg/mL (1.95 μM), 5 μg/mL (9.75 μM) or 10 μg/mL (19.5 μM). Humeral heads were stored at 4° C., protected from light and subjected to weekly media changes. Tissue was stored for 0 (fresh controls, n=2), 42 [DOX1 (n=4), DOX10 (n=4), DOX5 (n=1)] or 56 days [DOX1 (n=1), DOX10 (n=1), DOX5 (n=3)], where n refers to the number of humeral heads. As described above, chondrocyte viability was assessed at the end of the assigned storage times with tissue pieces processed for biochemical characterization of the extracellular matrix and histology, respectively (FIG. 20).

FIG. 20 is a schematic diagram showing the staining and imaging of humeral heads and subsequent analyses according to Example 3. Osteochondral samples were isolated from the humeral head, stained in Calcein AM and Ethidium Homodimer-1 and imaged on an inverted confocal microscope for live/dead quantification. One half of the humeral head processed for histological staining in safranin-O/fast green or toluidine blue for proteoglycan content. The other half was frozen and used for biochemical analyses of the extracellular matrix and cell number.

### Results

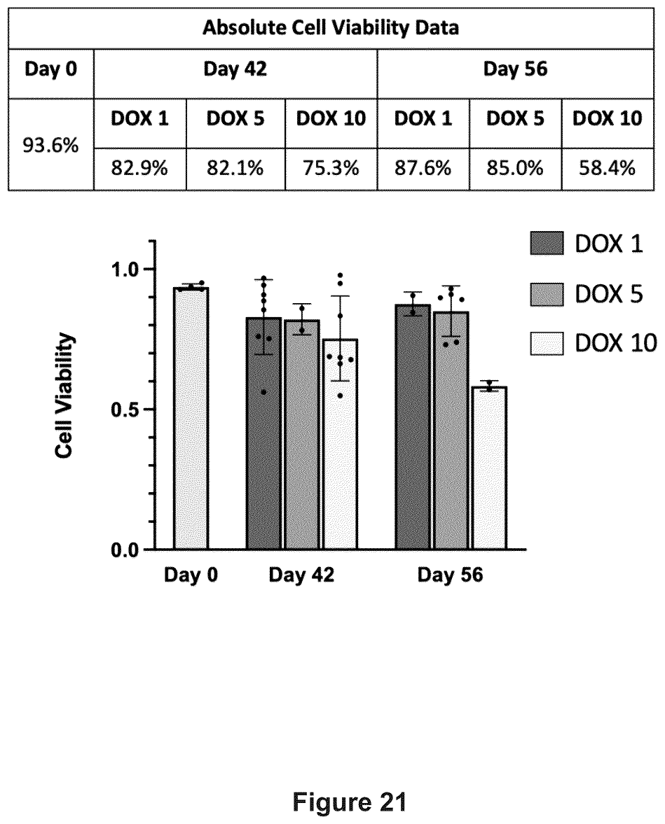

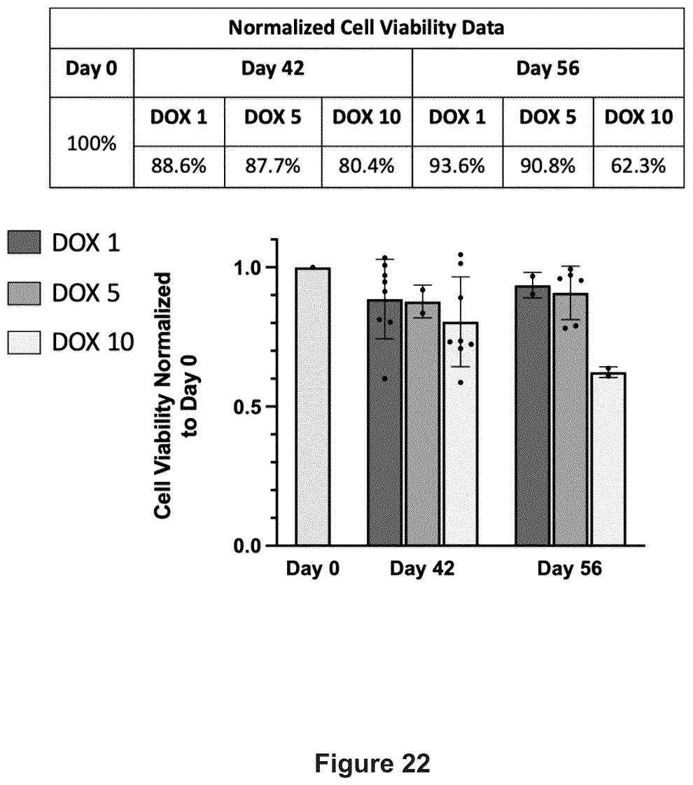

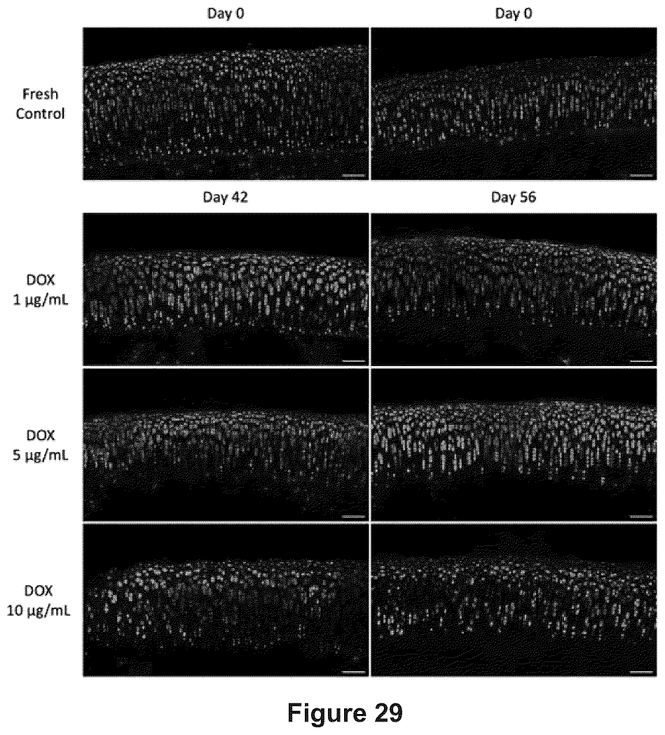

The effects of doxycycline concentration on chondrocyte viability were investigated using lapine humeral heads stored in DMEM (1.0 g/L glucose, L-glutamine, sodium pyruvate), high molecular weight hyaluronic acid (HA) (0.1% vol/vol) and doxycycline (DOX) at concentrations of either 1 μg/mL (DOX1), 5 μg/mL (DOX5) or 10 μg/m L (DOX10). Absolute cell viability data at the 42- and 56-day time points for the DOX1 and DOX5 formulations were similar at 82.9±13.4% and 82.1±5.5%, and higher than viability in the DOX10 formulation, which was 58.3±1.8% by the 56-day time point (FIGS. 21, 22). When normalized to fresh controls (93.6±1.1% viability), both DOX1 and DOX5 formulations maintained cell viability above 80% and above 90% after 42 and 56 days of storage, respectively (FIGS. 21, 22). There were no statistically significant differences between DOX1 and DOX5 at either Day 42 (p=0.935) or Day 56 (p=0.718), supporting the use of DOX5 in the DMEM-based media formulation to maximize its antibiotic effects without negatively affecting chondrocyte viability. At day 56, the results were statistically significantly different between cell viability in both DOX1 and DOX10 (p=0.036) and DOX5 and DOX10 (p=0.005). The distribution of live and dead chondrocytes (FIG. 29) shows that storage in DOX10 results primarily in dead cells being concentrated at the articular surface after both 42 and 56 days. This result indicates that both hyaluronic acid (HA) and the correct concentration of doxycycline (DOX) are required to adequately protect viability at the articular surface.

FIG. 21 is a graph of the absolute cell viability for different doxycycline concentrations over time, according to Example 3. FIG. 22 is a graph of the normalized cell viability for different doxycycline concentrations over time, according to Example 3. FIG. 29 shows confocal microscopy images of humeral head sections stored in varying concentrations of doxycycline (DOX) for 42 and 56 days. The distribution of live and dead chondrocytes is shown by Calcein AM positive (green) and Ethidium Homodimer-1 EtHD positive (red) cells. Images are oriented with the articular surface at the top and underlying subchondral bone at the bottom. The scale bar represents 100 μm.

### Example 4: The Inclusion of Dmem Improves Maintenance of Chondrocytes

### Methods

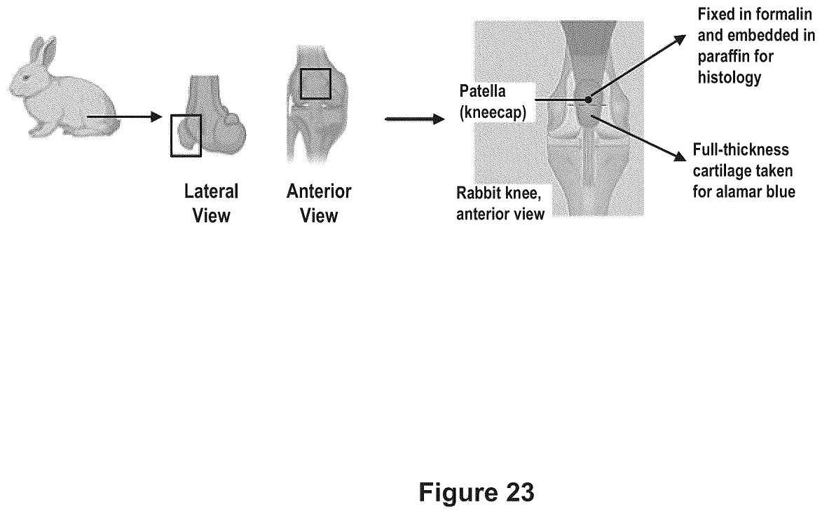

The patellae from 5 rabbits were isolated aseptically and used to evaluate the effects of DMEM as a component of the DMEM-based media formulation by comparing to LRS. The right patellae were stored in the DMEM-based media formulation consisting of DMEM (1.0 g/L glucose, L-glutamine, sodium pyruvate), hyaluronic acid (0.1% vol/vol) and doxycycline (5 μg/mL or 9.75 μM/mL) and the left patellae were stored in LRS, hyaluronic acid (0.1% vol/vol) and doxycycline (5 μg/mL). Samples were stored in 25 mL of their respective media for 0 (fresh control, n=3), 14 (n=1) or 28 days (n=3) at 4° C., protected from light and subjected to weekly media changes. At the end of the storage time, each patella was cut in half (FIG. 23) with one piece used to quantify chondrocyte metabolism and the other processed for histological evaluation, as described above.

FIG. 23 is a schematic diagram of the analyses for patellar cartilage, according to Example 4. Full-thickness cartilage was removed from one half of the patella for the Alamar blue assay to quantify chondrocyte metabolism. The remaining half of the patella underwent histological processing.

### Results

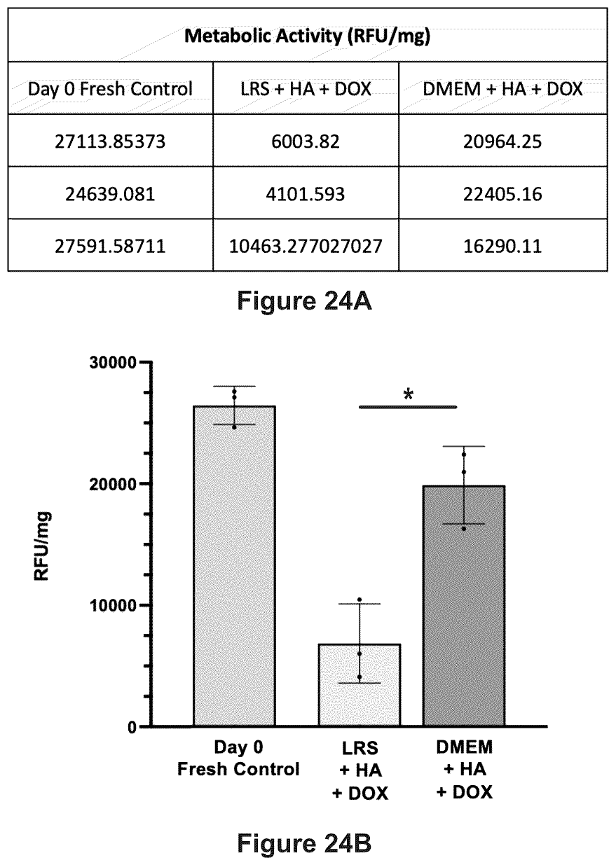

The effect of DMEM as a component of the DMEM-based media formulation was evaluated in comparison to LRS by storing lapine patellae in either the DMEM-based media consisting of DMEM (1.0 g/L glucose, L-glutamine, sodium pyruvate), hyaluronic acid (0.1% vol/vol) and doxycycline (5 μg/mL) or LRS supplemented with hyaluronic acid (0.1% vol/vol) and doxycycline (5 μg/mL). Average metabolic activity, assessed after 28 days in storage, was significantly greater (p=0.008) in samples stored in media containing DMEM, 19,886.5 RFU/mg, compared those stored in LRS, 6,856.2 RFU/mg (FIGS. 24A, 24B). These data support the inclusion of DMEM in the DMEM-based medial formulation as it suggests DMEM provides an environment that is more favorable to chondrocytes.

FIG. 24A is a table showing the metabolic activity of chondrocytes according to Example 4. FIG. 24B is a graph showing the metabolic activity of chondrocytes according to Example 4. Data are relative fluorescence units normalized to wet weight (RFU/mg) and bars are mean and standard deviation.

### Example 5: The Inclusion of Hyaluronic Acid Reduces Apoptotic Cells and does not Affect Total Metabolic Activity

### Methods

The patellae from 3 additional rabbits (5 patellae total) were isolated aseptically and used to investigate the effect of hyaluronic acid (HA) on chondrocyte metabolism. Patellae were stored in DMEM (1.0 g/L glucose, L-glutamine, sodium pyruvate) and doxycycline (5 μg/mL or 9.75 μM/mL) alone for 28 days (n=5). These samples were compared to the patellae used for Example 4, which were stored in DMEM (1.0 g/L glucose, L-glutamine, sodium pyruvate), doxycycline (5 μg/mL) and hyaluronic acid (0.1% vol/vol). Samples were stored at 4° C., protected from light and subjected to weekly media changes. After 28 days, each patella was cut in half (FIG. 23) with one piece used to quantify chondrocyte metabolism and measurement of chondrocyte number. Chondrocyte number was determined using the Hoechst 33258 fluorometric assay, which quantifies the amount of DNA in papain-digested cartilage samples mixed with the fluorescent dye Hoechst 33258. Hoechst 33258 is a bisbenzamine DNA intercalator that has an excitation maximum of 356 nm and an emission of 458 nm when bound to DNA. Specifically, the Hoechst 33258 dye binds to adenosine and thymidine rich regions of double-stranded DNA. The other half of the patella was processed for histological evaluation, as described above. Unstained, 5 μm histological sections were submitted for terminal deoxynucleotidyl transferase dUTP nick-end labeling (TUNEL) staining, to quantify apoptotic chondrocytes. The TUNEL assay detects double stranded DNA breaks resulting from the late phases of apoptosis.

### Results

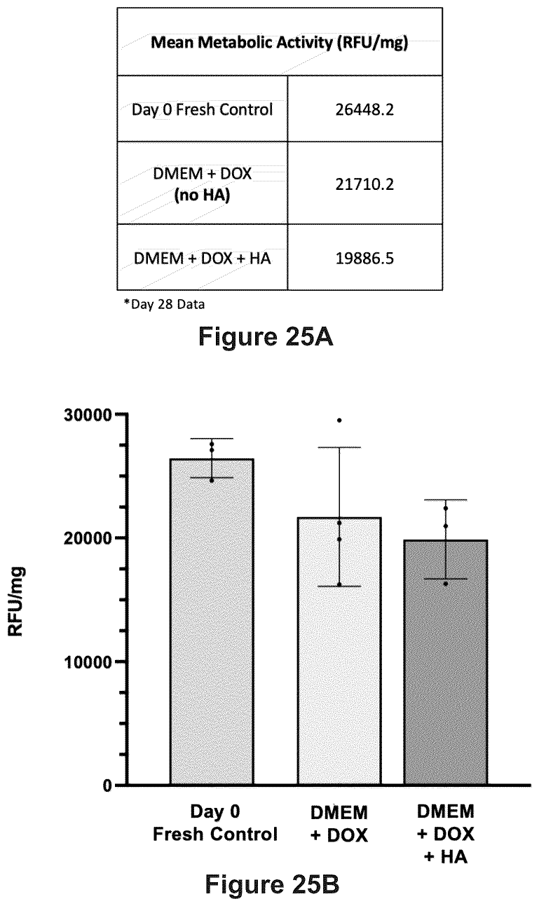

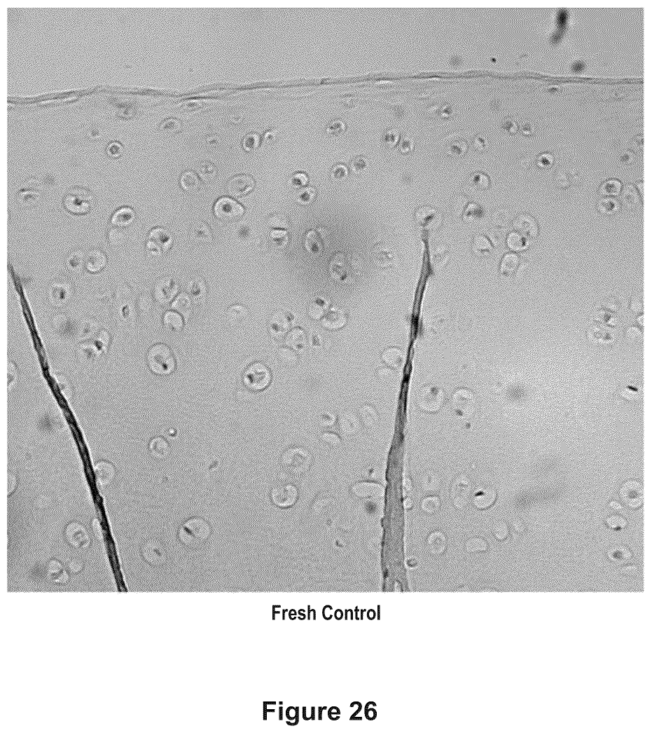

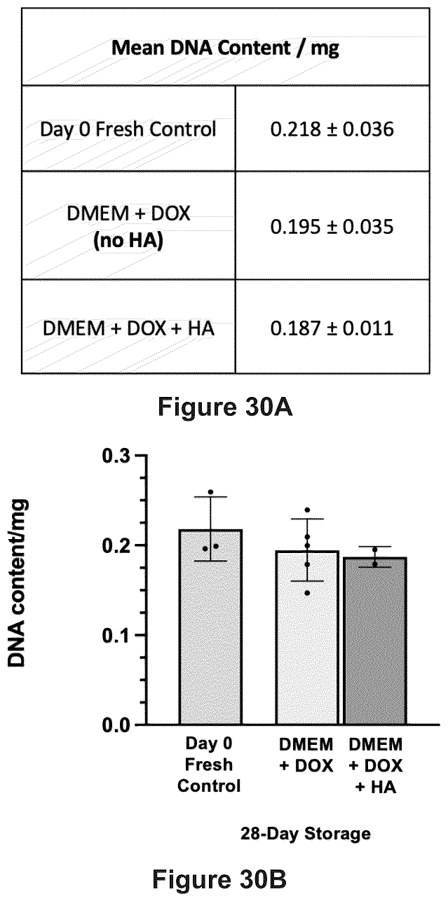

The contribution of hyaluronic acid (HA) to the performance of the DMEM-based media was investigated by storing lapine patellae in either DMEM (1.0 g/L glucose, L-glutamine, sodium pyruvate) and doxycycline (5 μg/mL) with or without hyaluronic acid (0.1% vol/vol). No difference in average metabolic activity (p=0.61) was detected after 28 days of storage with 19,886.5 RFU/mg measured in the solution containing hyaluronic acid (HA) and 20,549.9 RFU/mg in the solution without hyaluronic acid (HA), as shown in FIGS. 25A and 25B. No difference in DNA content (p=0.679), measured using the Hoechst 33258 assay, was detected (FIGS. 30A, 30B). TUNEL staining identified approximately 6% and 11% apoptotic chondrocytes in samples stored with and without hyaluronic acid (HA), respectively. These were both higher than the approximately 1.7% TUNEL positive cells measured in fresh controls (FIGS. 26-28). These data support the inclusion of hyaluronic acid in the DMEM-based media formulation as it has no effect on total metabolic activity nor cell count but reduces the number of apoptotic cells.

FIGS. 25A and 25B show chondrocyte metabolism measured in samples stored in DMEM (1.0 g/L glucose, L-glutamine, sodium pyruvate) and doxycycline (5 μg/mL) with or without hyaluronic acid (0.1% vol/vol). Data are relative fluorescence units normalized to wet weight (RFU/mg) and bars are mean and standard deviation.

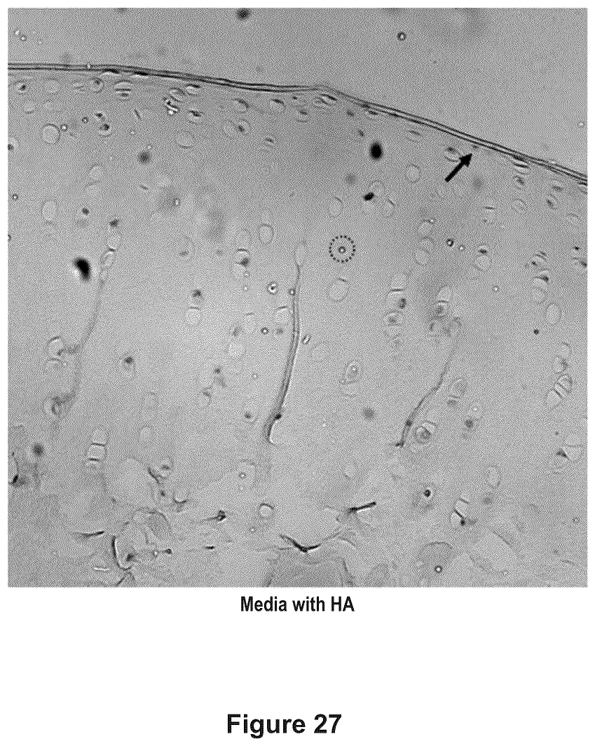

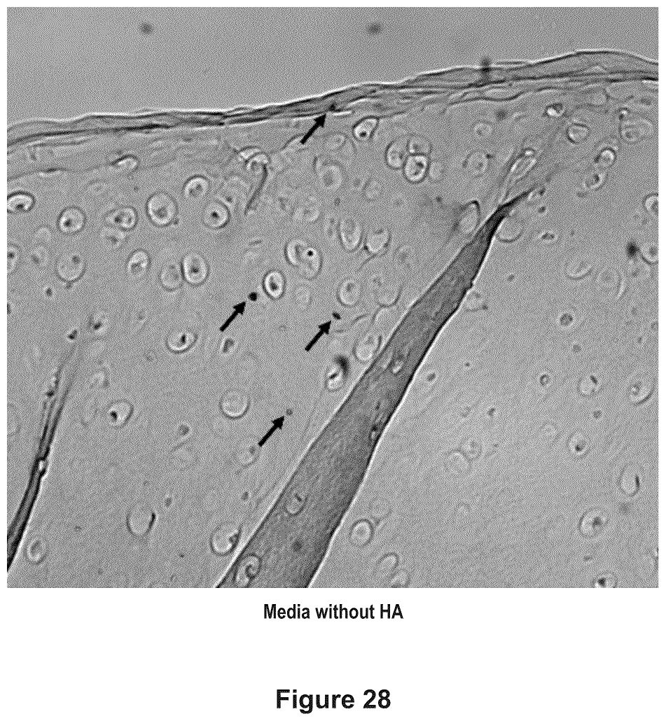

FIGS. 26-28 are representative TUNEL-stained images comparing media with hyaluronic acid (FIG. 27) and without hyaluronic acid (FIG. 28) to a fresh control (FIG. 26). Arrows point to TUNEL positive cells identifying apoptotic cells. The dotted circle identifies a processing artefact which is prevalent on this particular section.

### CONCLUSIONS

Examples 1-5 illustrate the efficacy of the DMEM-based media formulation, which consists of DMEM (1.0 g/L glucose, L-glutamine, sodium pyruvate), hyaluronic acid (0.1% vol/vol) and doxycycline (5 μg/mL). These three components work together to maintain chondrocyte viability and metabolism at levels that exceed the performance of LRS and, importantly, double the time when osteochondral tissues meet or exceed the 70% viability threshold for transplantation. Additionally, the DMEM-based media was found to have no detrimental effect on the cartilage extracellular matrix. The DMEM-based media is a highly effective method for preservation of osteochondral tissues that has the potential to significantly improve the supply of viable tissues for osteochondral allograft transplantation.

Since the DMEM media formulation was tested in rabbits, greater longevity is predicted when human tissue is stored in the media formulation.

### Example 6

A further study is proposed to thoroughly investigate the DMEM-based media with specific objectives to assess performance on prolonged storage of fresh human osteochondral tissues.

### Methods

A benchtop study using 12 cadaveric human distal femurs obtained from 6 donors of both sexes, aged 18 to 40 with normal Body Mass Index. Upon Institutional Review Board approval, these tissues will be obtained through our institutional bone bank (Mount Sinai Allograft Technologies, Toronto, Canada), which coordinates with the Trillium Gift of Life Network, a provincial organ and tissue donation network in Ontario, Canada. Obtaining tissues from enough donors may take several months and every effort will be made to use tissues consented only for research to minimize the number of donors diverted from clinical use. If we are unable to obtain enough donors in this way, tissue from other tissue banks will be requested.

The left distal femur from each donor will be assigned to storage in the optimized DMEM-based media formulation determined in Example 2 while the right distal femur will be stored in the LRS standard media as a control. All samples will be stored at 4° C. Each distal femur will be quartered to generate 4 osteochondral pieces assigned to storage times of 0 (fresh control), 42, 56, or 70 days of storage. Storage duration times and media change frequency may be adjusted. A group size of 6 (per storage condition and time point) was calculated to achieve a power of 80% by using means and variability obtained in the preliminary study. End-point analyses will include an assessment of chondrocyte viability and metabolism as described above. Effects on the cartilage ECM and bone quality will also be characterized using biochemical assays, histology, electromechanical measurements and biomechanical indentation testing.

Statistical comparisons will be made with a two-way ANOVA and Tukey's post hoc test. Independent variables include Storage Media (DMEM-based media, LRS standard media) and Storage Time (Day 0 fresh control, 42 days, 56 days and 70 days) with Dependent variables including chondrocyte viability, biochemical composition, apoptotic cell counts, chondrocyte metabolism, electromechanical measurements and biomechanical parameters. Sex will be reported even though the limited numbers of male and female donors may preclude statistically meaningful disaggregated data. Ethnicity is recognized to impact cartilage properties whereas gender is not anticipated to influence dependent variables. Both gender and ethnicity will be included in data collection when available in the donor records. Outliers will be identified as data points greater than two standard deviations from the mean. These experiments validate the performance of the DMEM-based media in human cartilage and will provide a foundation for collaboration with tissue banks to translate to clinical use. Studies involving human tissues will be conducted in compliance with good laboratory practices to support regulatory filing.

### Commentary

The performance of the optimized storage protocol in human cartilage may be influenced by species-specific differences. Compared to humans, rabbits have similar chondrocyte size but exhibit thinner cartilage, higher cell volume density and higher metabolic activity. In contrast, rabbits and humans share similar subchondral bone plate thickness, calcified cartilage thickness and bone mineral density. Consequently, osteochondral tissue isolated from a human is likely to be viable beyond 56 days. Sheep would be an alternative animal model.

The many features and advantages of the invention are apparent from the detailed specification and, thus, it is intended by the appended claims to cover all such features and advantages of the invention that fall within the true spirit and scope of the invention. Further, since numerous modifications and changes will readily occur to those skilled in the art, it is not desired to limit the invention to the exact construction and operation illustrated and described, and accordingly all suitable modifications and equivalents may be resorted to, falling within the scope of the invention.

What is claimed is:

1. A media formulation for preserving a biological sample comprising chondrocytes, the media formulation comprising Dulbecco's Modified Eagle Medium (DMEM), doxycycline, and hyaluronic acid,
wherein the doxycycline and sodium hyaluronate are present in the media formulation in amounts effective to prolong the viability of chondrocytes in a biological sample maintained in the media formulation.

2. The media formulation of claim 1 wherein the sodium hyaluronate has a molecular weight between about 1500 and about 2000 kDa.

3. The media formulation of claim 1 wherein the sodium hyaluronate comprises about 0.1 percent of the media formulation by volume.

4. The media formulation of claim 1 wherein the DMEM comprises between about 0 g/L and about 5 g/L glucose.

5. The media formulation of claim 3 wherein the DMEM comprises about 1 g/L glucose.

6. The media formulation of claim 1 wherein the DMEM comprises L-glutamine.

7. The media formulation of claim 1 wherein the DMEM comprises sodium pyruvate.

8. The media formulation of claim 1 wherein the doxycycline comprises about 1 μg/mL of the media formulation.

9. The media formulation of claim 1 wherein the doxycycline comprises about 5 μg/mL of the media formulation.

10. The media formulation of claim 1 wherein the doxycycline and sodium hyaluronate are present in the media formulation in amounts effective to maintain about 70% chondrocyte viability after a storage period of about 56 days.

11. A method comprising:
contacting a biological sample comprising chondrocytes with a media formulation comprising Dulbecco's Modified Eagle Medium (DMEM), doxycycline, and hyaluronic acid in amounts sufficient to prolong the viability of the chondrocytes; and
maintaining the biological sample in the media formulation at a storage temperature between about 1° and about 12° C., wherein at least 70% of chondrocytes in the biological sample remain viable after storage in the media formulation for a period of about 56 days.

12. The method of claim 11, wherein the storage temperature is about 10° C.

13. The method of claim 11, wherein the storage temperature is about 4° C.

14. The method of claim 11 further comprising harvesting the biological sample from a cadaveric donor.

15. The method of claim 14 wherein the biological sample is suitable for transplantation into a recipient.

16. The method of claim 15 wherein the biological sample comprises an osteochondral tissue.

17. The method of claim 16 further comprising assessing the viability of the chondrocytes, and if the viability exceeds a pre-determined threshold, transplanting the biological sample into the recipient.

18. The method of claim 17 wherein transplanting the biological sample into the recipient comprises press-fitting the biological sample into a recipient site.
